(19) United States
(12) Patent Application Publication
(10) Pub. No.: US 2022/0044829 A1
(43) Pub. Date: Feb. 10, 2022

(54) INTERACTIVE USER INTERFACE FOR PROVIDING TREATMENT INFORMATION

(71) Applicant: Cipherome, Inc., San Jose, CA (US)

(72) Inventors: Chan Hee Park, Seoul (KR); Joo Young Hong, Seoul (KR)

(21) Appl. No.: 17/396,324

(22) Filed: Aug. 6, 2021

Related U.S. Application Data
(60) Provisional application No. 63/062,992, filed on Aug. 7, 2020.

(51) Int. Cl. G16H 70/40; G16H 20/10; G16H 70/20; G16H 70/60; G16H 50/30

(52) U.S. Cl. CPC G16H 70/40; G16H 20/10; G16H 50/30; G16H 70/60; G16H 70/20

(57) ABSTRACT

A response prediction system uses sequenced genomic information obtained from the patient to generate patient specific treatment information. As an example, the treatment information can describe predicted dysfunction of proteins of the patient which can be targeted by a therapeutic as well as the safety, efficacy, or other effects of different drugs that can be administered to the patient. The response prediction system generates and provides an interactive user interface that includes the treatment information for presentation to a user, such as a physician. Altogether, the user can interact with the provided user interface to access the personalized treatment information.

### CROSS-REFERENCE TO RELATED APPLICATIONS

This application claims the benefit of U.S. Provisional Patent Application No. 63/062,992, filed on Aug. 7, 2020, which is incorporated herein in its entirety.

### BACKGROUND

### Field

The invention generally relates to information displayed through a user interface, and more specifically to an interactive user interface for displaying treatment related information for a patient.

### Background

Physicians are often tasked with the responsibility of monitoring numerous patients that suffer from various ailments at any one time. To understand possible treatment options, physicians can access conventional resources, such as online databases, to gain information about possible therapeutics that can be administered to treat a patient. However, these conventional resources provide details of treatment options at a general level and do not consider the possible unique effects that a treatment can have on a specific patient based on the patient's genetic makeup.

Additionally, there exist various, disparate conventional databases, each of which is cumbersome for a physician to search through to identify relevant treatment information for a specific therapeutic. The overwhelming nature of available treatment information across conventional databases is problematic because a physician can unintentionally receive and act upon incorrect treatment information.

### SUMMARY

Embodiments of the invention describe a centralized response prediction system that provides relevant treatment information that is personalized for a patient. Embodiments of the invention describe the presentation of the treatment information through interactive user interfaces (UIs). Therefore, if an interactive UI receives an input (e.g., from a physician), a different interactive UI that includes additional treatment information can be provided for display. By using the interactive UIs, a physician can conveniently access treatment information for a patient and undertake the appropriate course of action.

Each interactive UI includes a personalized representation for the patient with patient-specific treatment information. A personalized representation for the patient refers to a particular display of treatment information related to one or more drugs for the patient. For example, the personalized representation can include details of pharmacokinetics pathways of the one or more drugs, pharmacodynamics pathways of the one or more drugs, anatomical organ(s) of the patient that are likely affected by the one or more drugs, comparison of treatment information for the patient to other treatment information for other patients, and impact of the one or more drugs on one or more proteins and/or genes of the patient.

Treatment information displayed through a UI can include treatment information that describes the predicted impact of a treatment for a treatment. For example, treatment information includes a drug score that indicates the safety, efficacy, or other data about a drug if administered to a patient that is specific to that patient based on the patient's own genetic variation information. As another example, the treatment information includes a protein damage score that represents the predicted dysfunction of a protein in the patient such that the dysfunctional protein can be targeted by a treatment. In some embodiments, treatment information displayed through the UI includes only one of drug score(s) or protein damage score(s). In other embodiments, both drug score(s) and protein damage score(s) are displayed through the interactive UI.

In various embodiments, treatment information further includes comparisons between the numerical scores that have been generated for the patient and numerical scores that have been generated for other patients. Such treatment information can be generated based on unique patient information, such as genomic sequence information obtained from the patient including the particular variations found in the genome of a given patient. Therefore, the treatment information is personalized for the patient.

An embodiment includes a computer-implemented method for displaying treatment information for a patient for one or more drugs. The method includes steps of receiving individual gene sequence information of the patient, obtaining or generating treatment information including a set of drug safety scores for a set of drugs or a set of protein damage scores, wherein a protein damage score indicates a predicted dysfunction of a respective protein in the patient based on the gene sequence information of the patient, and wherein a drug safety score indicates a predicted adverse reaction to a respective drug for the patient based on the protein damage scores for proteins associated with pharmacokinetic (PK) or pharmacodynamic (PD) pathway of the respective drug, receiving, from a client device, a request for an interactive user interface providing information on administering the one or more drugs to the patient, generating instructions for display of a first screen of the interactive user interface. The first screen may be configured to display a visual depiction of a set of anatomical organs of a human body involved in pharmacokinetic (PK) pathways or pharmacodynamic (PD) pathways of the one or more drugs, and a set of interactive elements on the first screen, wherein each interactive element is displayed in association with a respective anatomical organ, and wherein the interactive element displays an indication of protein damage scores or drug safety scores based on the treatment information. The steps further include providing, to the client device for display, the first screen of the interactive user interface.

In one embodiment, the treatment information may include at least the set of drug safety scores, and wherein each interactive element is a graphical bar having a length proportional to a combination of one or more drug safety scores for the one or more drugs.

In one embodiment, the set of anatomical organs may each be assigned a respective color, and each interactive element may be displayed with a color assigned to the anatomial organ in association with the interactive element.

In one embodiment, the treatment information may include at least the set of protein damage scores, and the set of interactive elements may represent a set of proteins affected by the one or more drugs, and each interactive element may be overlaid on the anatomical organ where the PK or PD pathway involving the protein of the interactive element occur.

In one embodiment, each interactive element may be displayed with the indication of a protein damage score for the protein represented by the interactive element.

In one embodiment, the method further includes generating a color graph mapping a range of protein damage scores to a range of colors, and the indication for the interactive element may be a respective color for the protein damage score for the interactive element obtained from the color graph.

In one embodiment, the set of anatomical organs involved in the PK or PD pathway may be anatomical organs related to administration, transportation, or metabolism of the one or more drugs in the patient's body.

In one embodiment, the first screen may further display another set of interactive elements representing the one or more drugs or metabolites generated from the one or more drugs, and each interactive element may be overlaid on the anatomical organ where the PK or PD pathway involving the one or more drugs of the interactive element occur.

In one embodiment, the first screen may further include a directed edge from a first interactive element representing a drug overlaid on a first anatomical organ where the drug is administered to a second interactive element representing the drug overlaid on a second anatomical organ where the drug is transported or metabolized.

In one embodiment, the method further includes responsive to receiving another request from the client device, generating a second screen of the interactive user interface. The second screen may include a color graph mapping a range of drug safety scores to a range of colors, and an indication marking the drug safety scores of the one or more drugs for the patient on the color graph.

In one embodiment, a protein damage score for a respective protein may be computed from one or more genome sequence variation scores for one or more genome sequence variations in the patient, each genome sequence variation score indicating a degree of genome sequence variation in the patient that causes change to structure and/or function of the respective protein.

In one embodiment, the method further includes responsive to receiving another request from the client device, generating a second screen of the interactive user interface. The second screen may further include a line graph mapping the drug safety scores of the one or more drugs for the patient, where the one or more drugs have a same classification under anatomical therapeutical classification (ATC) system.

In one embodiment, the first screen may further display a visual depiction of protein damage scores, drug safety scores or their distribution in a population obtained using gene sequence information of the population.

In one embodiment, the first screen may further display a visual representation of the protein damage scores or the drug safety scores of the patient, relative to the protein damage scores, drug safety scores, or their distribution in the population.

In one embodiment, the population may represent an ethnic, racial, gender, or age group.

Another embodiment includes a system for displaying treatment information for a patient for one or more drugs. The system includes a processor and a computer readable storage medium for storing instructions executable by the processor. The instructions include receiving individual gene sequence information of the patient, obtaining or generating treatment information including a set of drug safety scores for a set of drugs or a set of protein damage scores, wherein a protein damage score indicates a predicted dysfunction of a respective protein in the patient based on the gene sequence information of the patient, and wherein a drug safety score indicates a predicted adverse reaction to a respective drug for the patient based on the protein damage scores for proteins associated with pharmacokinetic (PK) or pharmacodynamic (PD) pathway of the respective drug, receiving, from a client device, a request for an interactive user interface providing information on administering the one or more drugs to the patient, generating instructions for display of a first screen of the interactive user interface. The first screen may be configured to display a visual depiction of a set of anatomical organs of a human body involved in pharmacokinetic (PK) pathways or pharmacodynamic (PD) pathways of the one or more drugs, and a set of interactive elements on the first screen, wherein each interactive element is displayed in association with a respective anatomical organ, and wherein the interactive element displays an indication of protein damage scores or drug safety scores based on the treatment information. The steps further comprise providing, to the client device for display, the first screen of the interactive user interface.

In one embodiment, the treatment information may include at least the set of drug safety scores, and wherein each interactive element is a graphical bar having a length proportional to a combination of one or more drug safety scores for the one or more drugs.

In one embodiment, the set of anatomical organs may each be assigned a respective color, and wherein each interactive element is displayed with a color assigned to the anatomial organ in association with the interactive element.

In one embodiment, the treatment information may include at least the set of protein damage scores, and the set of interactive elements may represent a set of proteins affected by the one or more drugs, and each interactive element may be overlaid on the anatomical organ where the PK or PD pathway involving the protein of the interactive element occur.

In one embodiment, each interactive element may be displayed with the indication of a protein damage score for the protein represented by the interactive element.

In one embodiment, the instructions may further include generating a color graph mapping a range of protein damage scores to a range of colors, and the indication for the interactive element may be a respective color for the protein damage score for the interactive element obtained from the color graph.

In one embodiment, the set of anatomical organs involved in the PK or PD pathway may be anatomical organs related to administration, transportation, or metabolism of the one or more drugs in the patient's body.

In one embodiment, the first screen may further display another set of interactive elements representing the one or more drugs or metabolites generated from the one or more drugs, and each interactive element may be overlaid on the anatomical organ where the PK or PD pathway involving the one or more drugs of the interactive element occur.

In one embodiment, the first screen may further include a directed edge from a first interactive element representing a drug overlaid on a first anatomical organ where the drug is administered to a second interactive element representing the drug overlaid on a second anatomical organ where the drug is transported or metabolized.

In one embodiment, the instructions further include responsive to receiving another request from the client device, generating a second screen of the interactive user interface. The second screen may display a color graph mapping a range of drug safety scores to a range of colors, and an indication marking the drug safety scores of the one or more drugs for the patient on the color graph.

In one embodiment, a protein damage score for a respective protein may be computed from one or more genome sequence variation scores for one or more genome sequence variations in the patient, and each genome sequence variation score may indicate a degree of genome sequence variation in the patient that causes change to structure and/or function of the respective protein.

In one embodiment, the instructions may further include, responsive to receiving another request from the client device, generating a second screen of the interactive user interface. The second screen may display a line graph mapping the drug safety scores of the one or more drugs for the patient, wherein the one or more drugs have a same classification under anatomical therapeutical classification (ATC) system.

In one embodiment, the first screen may further display a visual depiction of protein damage scores, drug safety scores or their distribution in a population obtained using gene sequence information of the population.

In one embodiment, the first screen may further display a visual representation of the protein damage scores or the drug safety scores of the patient, relative to the protein damage scores, drug safety scores, or their distribution in the population.

In one embodiment, the population may represent an ethnic, racial, gender, or age group.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present invention will become better understood with regard to the following description, and accompanying drawings, where:

FIG. 1 is an overall system environment for providing an interactive UI for displaying treatment related information, in accordance with an embodiment.

FIG. 2A is an example UI that depicts a welcome screen, in accordance with an embodiment.

FIG. 2B is an example patient identification UI that depicts patient identification information, in accordance with an embodiment.

FIG. 3 is an example UI that depicts treatment information for a patient corresponding to one drug class, in accordance with an embodiment.

FIG. 4 is an example UI that depicts treatment information for a patient corresponding to multiple drug classes, in accordance with an embodiment.

FIG. 5 is an example UI that depicts additional details for drugs of a drug class, in accordance with an embodiment.

FIG. 6 is an example UI that depicts clinical details for a drug of a drug class, in accordance with an embodiment.

FIG. 7 is an example UI that depicts genetic information related to one drug of a drug class, in accordance with an embodiment.

FIG. 8 is an example UI that depicts an enlarged view of the panel depicting the genes affected by a drug information, in accordance with an embodiment.

FIG. 9 is an example UI that depicts pharmacokinetic (PK) pathways for a drug, in accordance with an embodiment.

FIG. 10 is an example UI that depicts an enlarged view of PK pathways for the drug, in accordance with an embodiment.

FIG. 11 is an example UI that depicts additional details of a drug overlaid with the PK pathways for the drug, in accordance with an embodiment.

FIG. 12 is an example UI that depicts additional details of a gene overlaid with the PK pathways for the drug, in accordance with an embodiment.

FIG. 13 is an example UI that depicts pharmacodynamics (PD) pathways for a drug, in accordance with an embodiment.

FIG. 14 is an example UI that depicts an enlarged view of PD pathways for the drug, in accordance with an embodiment.

FIG. 15 is an example UI that depicts additional details of a drug overlaid with the PD pathways for the drug, in accordance with an embodiment.

FIG. 16 is an example UI that depicts drug scores of varying drugs in a first category based on the anatomical therapeutic chemical (ATC) classification system, in accordance with an embodiment.

FIG. 17 is an example UI that depicts drug scores of varying drugs in a second category based on the ATC classification system, in accordance with an embodiment.

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them below.

The term “gene sequence variation information” used herein means information about substitution, addition, or deletion of a base constituting an exon of a gene. Such substitution, addition, or deletion of the base may result from various causes, for example, structural differences including mutation, breakage, deletion, duplication, inversion, and/or translocation of a chromosome or portion of a chromosome.

The term “gene sequence variation score” used herein refers to a numerical score of a degree of the individual genome sequence variation that causes an amino acid sequence variation (substitution, addition, or deletion) of a protein encoded by a gene or a transcription control variation and thus causes a significant change or damage to a structure and/or function of the protein when the genome sequence variation is found in an exon region of the gene encoding the protein. The gene sequence variation score can be calculated considering a degree of evolutionary conservation of amino acid in a genome sequence, a degree of an effect of a physical characteristic of modified amino acid on a structure or function of the corresponding protein.

The term “protein damage score” used herein refers to a numerical score calculated based on gene sequence variation scores. In one embodiment, if there is a single significant sequence variation in the gene region encoding the protein, a gene sequence variation score is identical to a protein damage score. In one embodiment, if there are two or more gene sequence variations encoding the protein, a protein damage score is calculated as a mean of gene sequence variation scores calculated for the respective variations.

The term “drug score” or “drug class score” used herein refers to a numerical score calculated with respect to a particular drug or a particular drug class, respectively, for an individual. The drug score or drug class score refers to the safety, efficacy, or other data about a drug or drug class, respectively for a patient. Determining the drug score or drug class score includes determining one or more target proteins involved in the pharmacodynamics or pharmacokinetics of the drug or drug class, such as an enzyme protein involved in drug metabolism, a transporter protein or a carrier protein. The drug score can be calculated based on protein damage scores of one or more genes encoding proteins involved in the pharmacodynamics or pharmacokinetics of the drug with respect to the individual. In some embodiments, the drug class score can be calculated based on the drug scores associated with drugs in the drug class.

### Overall System Environment

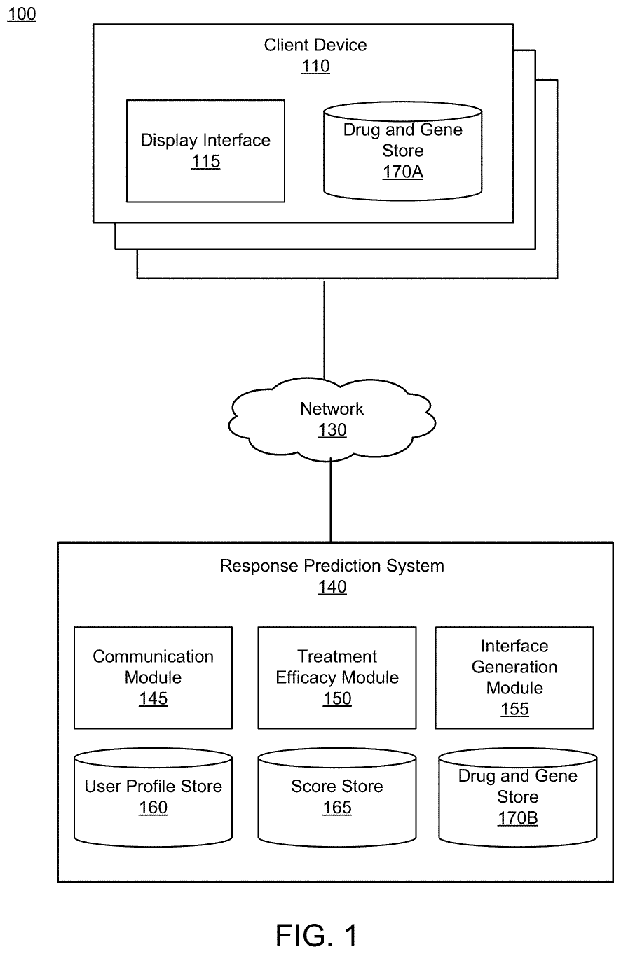

FIG. 1 is an overall system environment 100 for providing an interactive UI for displaying treatment related information, in accordance with an embodiment. The system environment 100 may include one or more client devices 110 and a response prediction system 140 connected to each other over a network 130. In other embodiments, different and/or additional entities can be included in the system environment.

Network

The network 130 facilitates communications between one or more client devices 110, and the response prediction system 140. The network 130 includes any combination of local area and/or wide area networks, using both wired and/or wireless communication systems. In one embodiment, the network 130 uses standard communications technologies and/or protocols. For example, the network 130 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 130 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 130 may be represented using any suitable format, such as hypertext markup language (HTML) or extensible markup language (XML). In some embodiments, all or some of the communication links of the network 130 may be encrypted using any suitable technique or techniques.

Client Device

The client device 110 is an electronic device such as a personal computer (PC), a desktop computer, a laptop computer, a notebook, a tablet PC executing an operating system, for example, a Microsoft Windows-compatible operating system (OS), Apple OS X, and/or a Linux distribution. In another embodiment, the client device 110 can be any device having computer functionality, such as a personal digital assistant (PDA), mobile telephone, smartphone, etc. The client device 110 transmits and receives data, such as patient information and/or treatment information, via the network 130.

As depicted in FIG. 1, the client device 110 can include a display interface 115 and a drug and gene store 170A. In one embodiment, the display interface 115 is embodied as a screen on the client device 110 such as a touch screen. In various embodiments, the client device 110 allows a user to provide inputs through the display interface 115 that can be transmitted through the network 130 to the response prediction system 140. For example, the client device 110 is capable of receiving user input from a user, such as a physician, through the display interface 115. A physician can provide patient information (e.g., patient registration number, date of birth, gender, genetic information of the patient, biomarker readings of the patient, past medical history of the patient, and the like) through the display interface 115 of the client device 110.

In some embodiments, a client device 110 executes an application allowing a user of the client device 110 to interact with the response prediction system 140. For example, a client device 110 executes an application to enable interaction between the client device 110 and the response prediction system 140 via the network 130 and to enable display of interactive UIs on the display interface 115. Such an application can be created by the response prediction system 140 and installed on the client device 110. In various embodiments, a user of the client device 110 creates login credentials (e.g., user identifier and password) using the application installed on the client device 110.

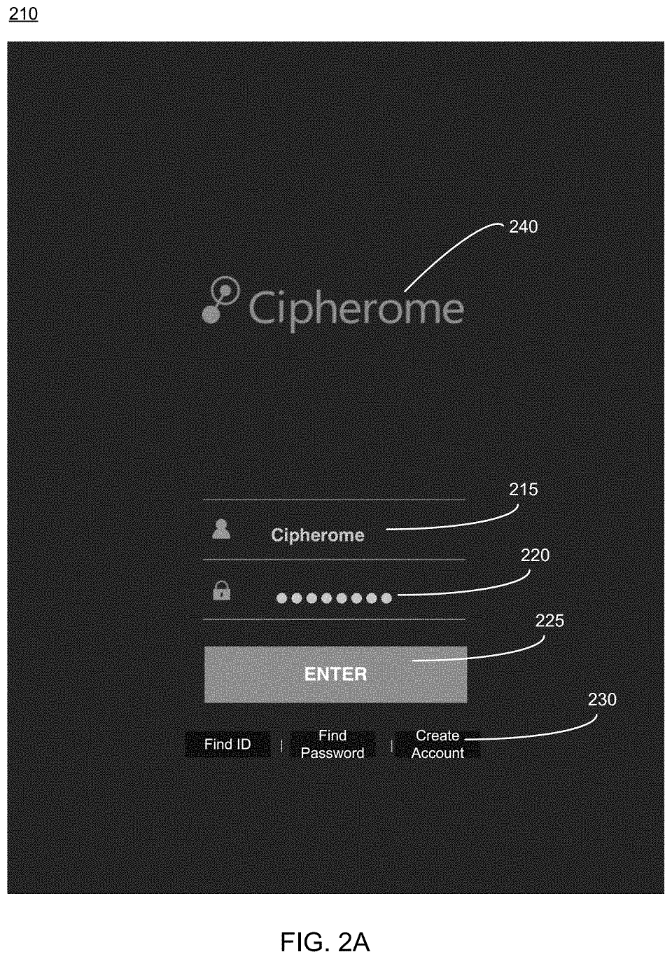

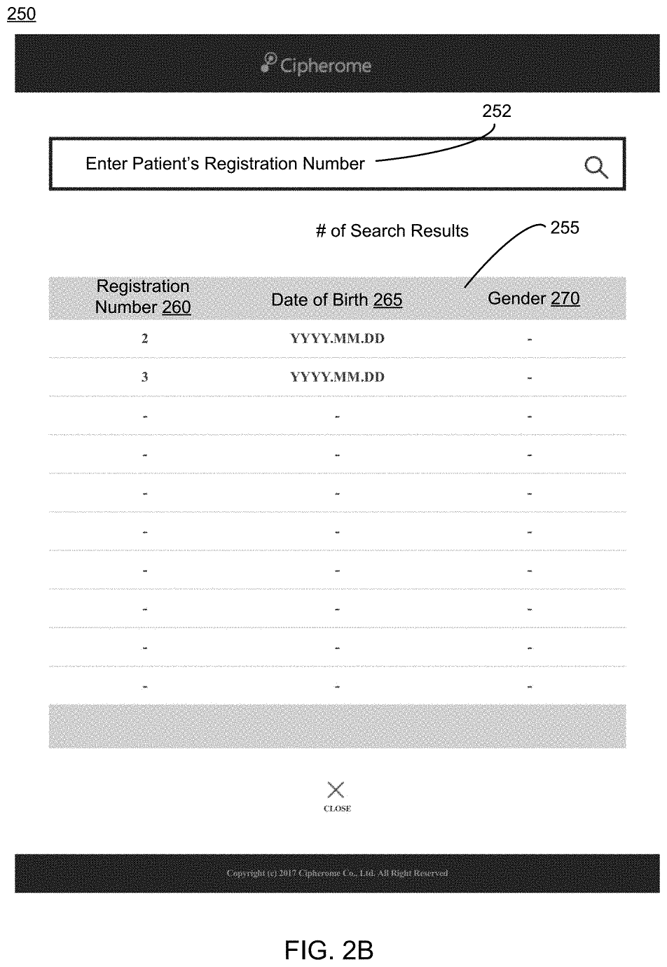

The display interface 115 can provide information received from the response prediction system 140 for display to a user of the client device 110. As an example, the display interface 115 can display an interactive UI, such as the UIs described below in relation to FIG. 2-17. In one embodiment, the interactive UI is generated by the response prediction system 140 and provided through the network 130 to the client device 110 for display on the display interface 115. In another embodiment, instructions for generating an interactive UI are provided from the response prediction system 140 through the network 130 to the client device 110. The client device 110 can generate the interactive UI using the received instructions. In some embodiments, the client device 110 combines treatment information received from the response prediction system 140 and combines the treatment information with details stored in the drug and gene store 170A to generate the interactive UI that can be presented by the display interface 115. As one example, the drug and gene store 170A can include graphical elements that are combined with treatment information calculated and transmitted from the response prediction system 140. Examples of graphical elements can include graphs, score bars, and pictures of anatomical structures, as will become evident in the interactive UIs depicted in FIG. 2-17. In various embodiments where the response prediction system 140 generates the interactive UI, the drug and gene store 170A can be solely embodied in the response prediction system 140 and is not included in the client device 110.

The display interface 115 can receive user interactions with an interactive UI displayed on the display interface 115. In various embodiments, in response to receiving a user interaction through the display interface, the client device 110 sends a request to the response prediction system 140 for a different interactive UI. Therefore, upon receipt of the different interactive UI, the display interface 115 can provide the different interactive UI for display. In some embodiments, the client device 110 receives more than one interactive UI such that upon receiving a user interaction on an interactive UI, the client device 110 can present a different interactive UI without having to send a request to the response prediction system 140.

Response Prediction System

The response prediction system 140 receives patient information and generates treatment information based on the received patient information. Subsequently, the generated treatment information can be arranged within an interactive UI that can be provided to a client device 110 for display. As an example, given the genomic sequencing information of the patient, the response prediction system 140 predicts treatment information for the patient such as the predicted dysfunction of a protein that can be targeted by a therapeutic or the safety of any one of a multitude of different drugs that can be administered to the patient. The treatment information of the patient can be assembled into an interactive UI that is transmitted to the client device 110 for presentation to a user of the client device 110. As shown in FIG. 1, the response prediction system 140 includes various modules such as a communication module 145, a treatment efficacy module 150, and an interface generation module 155. Additionally, the response prediction system 140 includes a user profile store 160, a score store 165, and a drug and gene store 170. In other embodiments, the response prediction system 140 may include additional, fewer, or different modules for various applications.

The communication module 145 controls communication between the response prediction system 140 and entities external to the response prediction system 140, such as communication over the network 130 with the client device 110. As one example, the communication module 145 receives user login credentials from the client device 110 and verifies the login credentials. To verify the login credentials, the communication module 145 can query the user profile store 160 that stores multiple user profiles. In various embodiments, each user profile is associated with a physician and therefore, user profile information can include patient information for the patients of the physician.

As another example, the communication module 145 can receive patient information for one or more patients to be used by the treatment efficacy module 150 for determining treatment options. In one embodiment, the communication module 145 receives patient information from the client device 110. In another embodiment, the communication module 145 receives patient information from a third party (e.g., a lab) that performs or maintains laboratory tests (e.g., coagulation test results or genomic sequence variation tests) for identifying patient information.

Patient information can refer to gene sequence information (e.g., nucleotide sequences) from a sample obtained from the patient. In one embodiment, the communication module 145 receives DNA sequences of the patient that can be used to identify gene sequence variation information. In some embodiments, the analysis of the patient's DNA sequences is conducted by a third party (e.g., a lab) such that the communication module 145 receives the gene sequence variation information from the third party. The communication module 145 provides the patient information to the treatment efficacy module 150 to be used to generate the treatment information.

Generally, the gene sequence variation information is information related to substitution, addition, or deletion of a nucleotide within an exon of a gene from the patient. In some embodiments, the substitution, addition, or deletion of the nucleotide results from breakage, deletion, duplication, inversion or translocation of a patient's chromosome or a portion of a chromosome. The genome sequence information of individuals used in the present invention may be determined by using a well-known sequencing method. Further, commercially available services such as those provided by Complete Genomics, BGI (Beijing Genome Institute), Knome, Macrogen, DNALink, etc. which provide commercialized services may be used, although not being limited thereto. Gene sequence variation information present in the genome sequence information of patients may be extracted by using various methods and may be acquired through sequence comparison analysis by using an algorithm such as ANNOVAR (Wang et al., Nucleic Acids Research, 2010; 38(16): e164), SVA (SequenceVariantAnalyzer) (Ge et al., Bioinformatics, 2011; 27(14): 1998-2000), BreakDancer (Chen et al., Nat Methods, 2009 September; 6(9): 677-81), etc., which compares a sequence to a reference group.

The communication module 145 transmits treatment information in an interactive UI to the client device 110 for display. The treatment information can be generated by the treatment efficacy module 150 and the interactive UI can be generated by the interface generation module 155. In some embodiments, the communication module 145 transmits an interactive UI generated by the interface generation module 155 through the network 130 to the client device 110. Here, the interactive UI includes treatment information generated by the treatment efficacy module 150, as is described in further detail below. In some embodiments, the communication module 145 transmits, to the client device 110, instructions generated by the interface generation module 155 along with the treatment information generated by the treatment efficacy module 150. In this scenario, the client device 110 generates the interactive UI that includes the treatment information based on the instructions. In various embodiments, the communication module 145 transmits one or more interactive UIs in response to a request from the client device 110.

Generating Treatment Information

The treatment efficacy module 150 generates treatment information for the patient based on the patient information, such as genomic sequences of the patient, received by the communication module 145. Treatment information refers to any of a gene sequence variation score, drug score, drug class score, and protein damage score, each of which is described in further detail below. In various embodiments, each score is a numerical value. Furthermore, in some embodiments, each score is normalized, such that it falls within a range (e.g., between 0 and 1).

In various embodiments, the treatment efficacy module 150 generates all possible scores (e.g., all possible gene sequence variation scores, drug scores for all possible drugs, drug class scores for all possible drug classes, and protein damage scores for all possible proteins) based on the received patient information. Therefore, when an interactive UI is needed, the treatment information needed for the interactive UI can be retrieved.

In various embodiments, the treatment efficacy module 150 generates treatment information in real-time based on the treatment information that is needed in a particular UI. For example, the communication module 145 may receive a request for a UI that is to be displayed with treatment information for a particular drug. Therefore, in response to the received request, the treatment efficacy module 150 can generate the drug score for that drug without generating drug scores for other drugs. Additionally, the treatment efficacy module 150 can identify proteins that are involved in the PD and PK pathways of that one drug and only generate protein damage scores for those identified proteins. The drug score for the particular drug and the protein damage scores for the identified proteins can then be provided to the interface generation module 155 to generate an interactive UI that is specific for displaying treatment information related to the one drug. When a new UI is needed that corresponds to a different drug (e.g., new request received by communication module 145), the treatment efficacy module 150 can generate a drug score for the different drug as well as protein damage scores for proteins that are involved in the PD and PK pathways for the different drug. By generating specific treatment information in real-time (e.g., based on the specific treatment information needed in an interactive UI), the treatment efficacy module 150 can avoid generating various treatment information that may not be provided for display.

Referring to the gene sequence variation score, the treatment efficacy module 150 generates one or more gene sequence variation scores using the gene sequence variation information for the patient. The treatment efficacy module 150 calculates a gene sequence variation score from the patient's gene sequence variation information using one or more algorithms selected from the group consisting of: SIFT (Sorting Intolerant From Tolerant), PolyPhen, PolyPhen-2 (Polymorphism Phenotyping), MAPP (Multivariate Analysis of Protein Polymorphism), Logre (Log R Pfam E-value), Mutation Assessor, Condel, GERP (Genomic Evolutionary Rate Profiling), CADD (Combined Annotation-Dependent Depletion), MutationTaster, MutationTaster2, PROVEAN, PMuit, CEO (Combinatorial Entropy Optimization), SNPeffect, fathmm, MSRV (Multiple Selection Rule Voting), Align-GVGD, DANN, Eigen, KGGSeq, LRT (Likelihood Ratio Test), MetaLR, MetaSVM, MutPred, PANTHER, Parepro, phastCons, PhD-SNP, phyloP, PON-P, PON-P2, SiPhy, SNAP, SNPs&GO, VEP (Variant Effect Predictor), VEST (Variant Effect Scoring Tool), SNAP2, CAROL, PaPI, Grantham, SInBaD, VAAST, REVEL, CHASM (Cancer-specific High-throughput Annotation of Somatic Mutations), mCluster, nsSNPAnayzer, SAAPpred, HanSa, CanPredict, FIS and BONGO (Bonds ON Graphs).

In various embodiments, the one or more gene sequence variations correspond to proteins that are involved in the pharmacodynamics (PD) or pharmacokinetics (PK) of a drug or drug group. These gene sequence variations can then be used to personalize the treatment information that is provided for the particular patient. For example, the gene sequence variation score can be used to further calculate the protein damage score for a protein that is involved in the PD or PK of a drug or drug group. In some embodiments, the protein damage score is calculated as a mean of the plurality of gene sequence variation scores. Additionally, the gene sequence variation score can be used to calculate a drug score for a drug or a drug class score for a drug class.

Referring to the generation of a protein damage score, as an example, the protein damage score can be calculated from the gene sequence variation information by using an algorithm such as SIFT (Sorting Intolerant From Tolerant, Pauline C et al., Genome Res. 2001 May; 11(5): 863-874; Pauline C et al., Genome Res. 2002 March; 12(3): 436-446; Jing Hul et al., Genome Biol. 2012; 13(2): R9), PolyPhen, PolyPhen-2 (Polymorphism Phenotyping, Ramensky V et al., Nucleic Acids Res. 2002 September 1; 30(17): 3894-3900); Adzhubei et al., Nat Methods 7(4): 248-249 (2010)), MAPP (Eric A. et al., Multivariate Analysis of Protein Polymorphism, Genome Research 2005; 15: 978-986), Logre (Log R Pfam E-value, Clifford R. J. et al., Bioinformatics 2004; 20: 006-1014), Mutation Assessor (Reva B et al., Genome Biol. 2007; 8: R232, http://mutatioassessor.org/), Condel (Gonzalez-Perez A et al., The American Journal of Human Genetics 2011; 88: 440-449, http://bg.upf.edu/fannsdb/), GERP (Cooper et al., Genomic Evolutionary Rate Profiling, Genome Res. 2005; 15; 901-913, http://mendel.stanford.edu/SidowLab/downloads/gerp/), CADD (Combined Annotation-Dependent Depletion, http://cadd.gs.washington.edu/), MutationTester, MutationTester2 (Schwarz et al., MutationTester2: mutation prediction for the deep-sequencing age. Nature Methods 2014; 11: 361-362, http://www.mutationtester.org/), PROVEAN (Choi et al., PLoS One 2012; 7(10): e46688), PMut (Ferrer-Costa et al., Proteins 2004; 57(4): 811-819, http://mmb.pcb.ub.es/PMut/), CEO (Combinatorial Entropy Optimization, Reva et al., Genome Biol. 2007; 8(11): R232), SNPeffect (Reumers et al., Bioinformatics 2006; 22(17): 2183-2185, http://snpeffect.vib.be), FATHMM (Shihab et al., Functional Analysis through Hidden Markov Models, Hum Mutat 2013; 34: 57-65, http://fathmm.biocompute.org.uk/), etc., although not being limited thereto.

The above-described algorithms are configured to identify how much each gene sequence variation has an effect on a protein function or whether or not there are any other effects. These algorithms have common aspects in that they are configured to consider an amino acid sequence of a protein encoded by a corresponding gene and relevant effects caused by an individual gene sequence variation that may lead to dysfunction on a structure and/or function of the corresponding protein.

As one example, the protein damage score is calculated by the following Equation 1. The following Equation 1 can be modified in various ways, and, thus, the present invention is not limited thereto.

$${S}_{g} ( {v}_{1} , … , {v}_{n} ) = {( \frac{1}{n} {\sum }_{i = 1}^{n} {v}_{i}^{p} )}^{\frac{1}{p}} \qquad ( 1 )$$

In Equation 1, S_{g} is a protein damage score of a protein encoded by a gene g, and n is the number of target sequence variations for analysis among sequence variations of the gene g, vᵢ is a gene sequence variation score of an i-th gene sequence variation, and p is a real number other than 0. In Equation 1, when a value of the p is 1, the protein damage score becomes an arithmetic mean, if the value of the p is −1, the protein damage score becomes a harmonic mean, and if the value of the p is close to the limit 0, the protein damage score becomes a geometric mean.

As another example, the protein damage score is calculated by the following Equation 2.

$${S}_{g} ( {v}_{1} , … , {v}_{n} ) = {( {\prod }_{i = 1}^{n} {v}_{i}^{{w}_{i}} )}^{\frac{1}{{\sum }_{i = 1}^{n} {w}_{i}}} \qquad ( 2 )$$

In Equation 2, S_{g} is a protein damage score of a protein encoded by a gene g, and n is the number of target sequence variations for analysis among sequence variations of the gene g, vᵢ is a gene sequence variation score of an i-th gene sequence variation, and wᵢ is a weighting assigned to the vᵢ. If all weightings wᵢ have the same value, the protein damage score S_{g} becomes a geometric mean of the gene sequence variation scores v₁. The weighting may be assigned considering a class of the corresponding protein, PD or PK classification of the corresponding protein, PK parameters of the enzyme protein of a corresponding drug, a population group, or a race distribution.

Referring to the generation of a drug score, it is calculated by associating the above-described protein damage score with a drug-protein relation. In one embodiment, if two or more proteins involved in the PD or PK of a particular drug or drugs are damaged, a drug score is calculated as a mean of the protein damage scores. Such a mean can be calculated, for example, as a geometric mean, an arithmetic mean, a harmonic mean, an arithmetic-geometric mean, an arithmetic-harmonic mean, a geometric-harmonic mean, a Pythagorean mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication or a weighted multiplication, or by a functional operation of the calculated values, although not being limited thereto.

The drug score may be calculated by adjusting weightings of a target protein involved in the PD or PK of the corresponding drug, an enzyme protein involved in drug metabolism, a transporter protein or a carrier protein in consideration of pharmacological characteristics, and the weighting may be assigned considering PK parameters of the enzyme protein of a corresponding drug, a population group, a race distribution, or the like. Further, although not directly interacting with the corresponding drug, proteins interacting with a precursor of the corresponding drug and metabolic products of the corresponding drug, for example, proteins involved in a pharmacological pathway, may be considered, and protein damage scores thereof may be combined to calculate the individual drug score. Further, protein damage scores of proteins significantly interacting with the proteins involved in the PD or PK of the corresponding drug may also be considered and combined to calculate the drug score. Information about proteins involved in a pharmacological pathway of the corresponding drug, which significantly interact with the proteins in the pathway or are involved in a signal transduction pathway thereof, can be identified from the drug and gene store 170B which can incorporate data from publicly known biological databases such as PharmGKB (Whirl-Carrillo et al., Clinical Pharmacology & Therapeutics 2012; 92(4): 414-4171), the MIPS Mammalian Protein-Protein Interaction Database (Pagel et al., Bioinformatics 2005; 21(6): 832-834), BIND (Bader et al., Biomolecular Interaction Network Database, Nucleic Acids Res. 2003 Jan. 1; 31(1): 248-50), Reactome (Joshi-Tope et al., Nucleic Acids Res. 2005 Jan. 1; 33 (Database issue): D428-32), etc.

In one embodiment, the drug score is calculated by the following Equation 3. The following Equation 3 can be modified in various ways, and, thus, the present invention is not limited thereto.

$${S}_{d} ( {g}_{1} , … , {g}_{n} ) = {( \frac{1}{n} {\sum }_{i = 1}^{n} {g}_{i}^{p} )}^{\frac{1}{p}} \qquad ( 3 )$$

In Equation 3, S_{d} is an individual drug score of a drug d, n is the number of proteins directly involved in the PD or PK of the drug d or interacting with a precursor of the corresponding drug or metabolic products of the corresponding drug, for example, proteins encoded by one or more genes selected from a gene group involved in a pharmacological pathway, gᵢ is a protein damage score of a protein directly involved in the PD or PK of the drug d or interacting with a precursor of the corresponding drug or metabolic products of the corresponding drug, for example, a protein encoded by one or more genes selected from a gene group involved in a pharmacological pathway, and p is a real number other than 0. In Equation 3, when a value of the p is 1, the drug score becomes an arithmetic mean, if the value of the p is −1, the drug score is becomes harmonic mean, and if the value of the p is close to the limit 0, the individual drug score becomes a geometric mean.

In another embodiment, the drug score is calculated by the following Equation 4.

$${S}_{d} ( {g}_{1} , … , {g}_{n} ) = {( {\prod }_{i = 1}^{n} {g}_{i}^{{w}_{i}} )}^{\frac{1}{{\sum }_{i = 1}^{n} {w}_{i}}} \qquad ( 4 )$$

In Equation 4, S_{d} is an individual drug score of a drug d, n is the number of proteins directly involved in the pharmacodynamics or pharmacokinetics of the drug d or interacting with a precursor of the corresponding drug or metabolic products of the corresponding drug, for example, proteins encoded by one or more genes selected from a gene group involved in a pharmacological pathway, g₁ is a protein damage score of a protein directly involved in the pharmacodynamics or pharmacokinetics of the drug d or interacting with a precursor of the corresponding drug or metabolic products of the corresponding drug, for example, a protein encoded by one or more genes selected from a gene group involved in a pharmacological pathway, and wᵢ is a weighting assigned to the gᵢ. If all weightings wᵢ have the same value, the individual drug score S_{d} becomes a geometric mean of the protein damage scores gᵢ. The weighting may be assigned considering the kind of the protein, the pharmacodynamic or pharmacokinetic classification of the protein, the pharmacokinetic parameters of the enzyme protein of the corresponding drug, a population group or a race distribution.

In the case of a geometric mean calculation method, weightings are equally assigned regardless of the characteristic of a drug-protein relation. However, it is possible to calculate a drug score by assigning weightings considering each characteristic of a drug-protein relation as described in yet another exemplary embodiment. For example, different scores may be assigned to a target protein of a drug and a transporter protein related to the drug. Further, it is possible to calculate an individual drug score by assigning the PK parameters Kₘ, Vₘₐₓ, and K_{cat}/Kₘ as weightings to the enzyme protein of a corresponding drug. Furthermore, for example, since a target protein is regarded more important than a transporter protein in terms of pharmacological action, it may be assigned a higher weighting, or a transporter protein or a carrier protein may be assigned high weightings with respect to a drug whose effectiveness is sensitive to a concentration, but the present invention is not limited thereto. The weighting may be minutely adjusted according to the characteristics of a relation between a drug and a protein related to the drug and the characteristics of an interaction between the drug and the protein. A sophisticated algorithm configured to assign a weighting considering the characteristic of an interaction between a drug and a protein can be used. For example, a target protein and a transporter protein may be assigned 2 points and 1 point, respectively.

Referring now to a drug class score, it can be calculated from the individual drug scores associated with drugs in the drug class. As an example, the individual drug scores of drugs in a particular drug class can be averaged to obtain the drug class score for the drug class. In some embodiments, individual drug scores are differently weighted for the determination of a drug class score. As an example, drugs of a drug class that are administered more frequently to patient populations can be weighted more heavily than drugs of the drug class that are administered less frequently.

Each of the gene sequence variation score, drug score, drug class score, and protein damage score can be stored in the score store 165.

In the above description, only the protein directly interacting with a drug has been exemplified. However, in various embodiments, the predictive ability of the above equations can be improved by using information about the protein interacting with a precursor of the corresponding drug or metabolic products of the corresponding drug, the protein significantly interacting with proteins involved in the PD or PK of the corresponding drug, and the protein involved in a signal transduction pathway thereof. By using information about a protein-protein interaction network or pharmacological pathway, it is possible to use information about various proteins relevant thereto. Therefore, even if a significant variation is not found in the protein directly interacting with the drug and thus there is no protein damage score calculated with respect to the protein or there is no damage (for example, 1.0 point when the SIFT algorithm is applied), a mean (for example, a geometric mean) of protein damage scores of proteins interacting with the protein or involved in the same signal transduction pathway of the protein may be used as a protein damage score of the protein so as to be used for calculating a drug score.

In various embodiments, the treatment efficacy module 150 further ranks the drug scores and drug class scores. As one example, the treatment efficacy module 150 ranks the drug scores and drug class scores in order to compare the safety of individual drugs and drug classes for a patient to other drugs and other drug classes, respectively. In various embodiments, the treatment efficacy module 150 compares the drug scores and drug class scores generated for a patient to drug scores and drug class scores that were generated for other patients. As an example, the treatment efficacy module 150 can retrieve drug and drug class scores from the score store 165 and determine a percentile ranking for each drug score for a patient in relation to drug scores for similar patients.

Generating Interactive User Interfaces

The interface generation module 155 generates interactive UIs or generates instructions that can be used to generate an interactive UI. In some embodiments, the interface generation module 155 generates an interactive UI and transmits the UI through the network 130 to the client device 110 such that the client device 110 can present the interactive UI to a user of the client device 110 through the display interface 115. More specifically, the interface generation module 155 generates interactive UIs that include the treatment information generated by the treatment efficacy module 150 as well as additional information from the drug and gene store 170B. In one embodiment, the interface generation module 155 generates visual graphs based on the calculated treatment information from the treatment efficacy module 150. For example, given the drug score for a patient, the interface generation module 155 can generate a visual graph depicting the drug score for the patient in relation to drug scores of other patients. Therefore, the visual graph can be included in the interactive UI that is transmitted to the client device 110.

In other embodiments, the interface generation module 155 generates instructions for generating a user interface that would incorporate the treatment information generated by the treatment efficacy module 150. As an example, instructions for generating a UI can include instructions as to how the client device 110 is to combine data stored on the client device 110 (e.g., data stored in the drug and gene store 170A) with treatment information generated by the treatment efficacy module 150 to generate the interactive UI that is to be displayed on the display interface 115. As another example, instructions for generating a UI can describe how the treatment information is to be displayed in the interactive UI (e.g., location in the UI, colors and/or shapes in the UI, order of appearance in the UI).

The drug and gene store 170B may store a variety of information for different drugs and genes that are related to the treatment information generated by the treatment efficacy module 150. As one example, the drug and gene store 170B stores classification information, such as anatomical therapeutic chemical (ATC) classification, for each drug in the drug and gene store 170B. The drug and gene store 170 may refer to one or more databases containing information about a protein involved in the PD or PK of a particular drug or drugs, for example, a gene encoding a target protein relevant to the drug, an enzyme protein involved in drug metabolism, a transporter protein, a carrier protein, etc.

As another example, the drug and gene store 170B can include drug information for each drug including, but not limited to, name of drug, possible side effects of a drug, possible interactions between the drug and other drugs, mode of action, dosing regimen, and the like. As another example, the drug and gene store 170B includes gene information for each gene including, but not limited to, name of gene, location of gene in the genome, protein that is translated from the gene, type of protein (e.g., enzyme) translated from the gene, target of the protein that is translated from the gene, and the like. As another example, the drug and gene store 170B can include drug and gene interactions. In some embodiments, the drug and gene store 170B includes drug and protein interactions, where the proteins are translated from a gene stored in the drug and gene store 170B.

The information stored in the drug and gene store 170B, such as drug information related to specific drugs, genes, drug-gene interactions, and drug-protein interactions, can be curated from public or non-public databases or a knowledge bases including, e.g., DrugBank (http://drugbank.ca/), KEGG DRUG (http://www.genome.jp/kegg/drug/), PharmGKB (http://www.pharmgkb.org/), etc., although not being limited thereto.

As described above in relation to drug and gene store 170A in the client device 110, the drug and gene store 170B of the response prediction system 140 can further include graphical elements such as graphs, score bars, and pictures of anatomical structures. Therefore, the interface generation module 155 can fully generate an interactive UI by accessing graphical elements stored in the drug and gene store 170B and combining the graphical elements with the treatment information (e.g., scores).

While FIG. 1 illustrates a system environment 100 in which a response prediction system 140 includes both a treatment efficacy module 150 for generating the treatment information and the interface generation module 155 for generating the interactive UI (which is then sent to a client device 110 via a communication module 145), it should be appreciated that the system environment 100 can include other types of configurations that enable the interactive UI to be generated at the client device 110 in conjunction with treatment information. For example, in one instance, the treatment efficacy module 150 may be included in a first server distinct from a second server including the interface generation module 155 and the communication module 145. Thus, one entity managing the first server may be responsible for generating the treatment information (e.g., drug safety scores, protein damage scores, rankings), while another separate entity managing the second server may be responsible for generating the actual interactive UI responsive to obtaining the treatment information for a patient from the first server. In such an embodiment, the second server may request treatment information related to a patient from the first server, and the first server may provide the treatment information back to the second server responsive to the request. The second server may then generate an interactive UI based on the treatment information and provide instructions for displaying the interactive UI to the client device 110.

Examples of interactive UIs that are either generated by the interface generation module 155 or generated by the client device 110 using instructions generated by the interface generation module 155 are described below in further detail in relation to FIG. 2-17.

### Example Interactive User Interfaces

Reference is now made to FIG. 2-17, each of which depicts an example interactive graphical UI displayed using the display interface 115 of a client device 110. Some descriptions hereafter will refer to particular transitions between a first interactive UI to a second interactive UI; however, in various embodiments, other transitions between UIs can be readily envisioned.

Generally, the interactive UI can include a personalized representation for the patient with patient-specific treatment information corresponding to one or more drugs. As one specific example, the personalized representation includes personalized treatment information such as the predicted safety or efficacy of a drug if administered to the patient. Additionally or alternatively, the personalized representation in the interactive UI provides details of predicted interactions between the drug and the patient's proteins and/or genes.

In one example, the interface generation module 155 can obtain treatment information including a set of protein damage scores or a set of drug safety scores (for a set of drugs) for a patient, for example, from the treatment efficacy module 150. The protein damage score may indicate a predicted dysfunction of a respective protein in the patient based on the gene sequence information of the patient. A drug safety score indicates a predicted adverse reaction to a respective drug for the patient based on the protein damage scores for proteins associated with PK or PD pathway of the respective drug.

Responsive to receiving a request from a client device for information on administering the one or more drugs to the patient, the interface generation module 155 may generate instructions for display of a first screen of the interactive UI. The first screen may be configured to display a visual depiction of a set of anatomical organs of a human body involved in PK pathways or PD pathways of the one or more drugs and a set of interactive elements on the first screen. Each interactive element may be displayed in association with a respective anatomical organ, and may display an indication of protein damage scores or drug safety scores based on the treatment information.

As described in more detail below, the interactive UI may switch to displaying other screens that display other types of information responsive to a user request. For example, the user request may be in the form of clicking a tab, clicking one or more interactive elements (e.g., nodes, graphs, graphical elements displayed on the interactive UI), and the like.

Introductory User Interfaces

FIG. 2A is an example UI that depicts a welcome screen 210, in accordance with an embodiment. In one embodiment, the welcome screen 210 is displayed on the display interface 115 in response to the execution of an application stored on the client device 110. Therefore, the welcome screen 210 is the first display provided to a user of the client device 110.

As shown in FIG. 2A, the welcome screen 210 is used to login to access data stored and generated by the response prediction system 140. The welcome screen 210 can include a user identifier field 215, a password field 220, a selectable submission key 225, and one or more selectable options 230 related to logging into the system. For example, the selectable options 230 can include an option to obtain a user identifier to be entered into the user identifier field 215, an option to obtain a password to be entered into the password field 220, and an option to create a new account (e.g., create a new user identifier and/or password). In various embodiments, the welcome screen 210 can further include identification of an entity 240 associated with the response prediction system 140.

FIG. 2B is an example patient identification UI 250 that depicts example patient identification information, in accordance with an embodiment. In various embodiments, the patient identification UI 250 is displayed in response to the selection of the submission key 225 (see FIG. 2A) and successful verification of the login credentials (e.g., user identifier and password). As shown in FIG. 2B, the example patient identification UI 250 includes a search query field 252 that enables the entering and searching of a patient who is assigned a patient registration number. The patient identification UI 250 can depict one or more patients as well as information related to the one or more patients in an array 255. For example, the array 255 may be indexed by the patient registration number 260, the patient date of birth 265, and/or the patient gender 270.

In various embodiments, each of the patients and patient information related to each patient shown in the array 255 are associated with a user profile corresponding to the login credentials that were used to login to the response prediction system 140. As described above in relation to the user profile store 160, a user profile may be associated with a physician. Therefore, each patient depicted in the array 255 are patients that are assigned to the physician. In some embodiments, as patient registration information is entered into the search query field 252, the array 255 can be updated to depict patients that match the entered information.

In various embodiments, each patient and patient information depicted in the array 255 (e.g., each row in the array 255) is selectable to obtain treatment information for the patient.

User Interface Depicting Treatment Information for a Patient

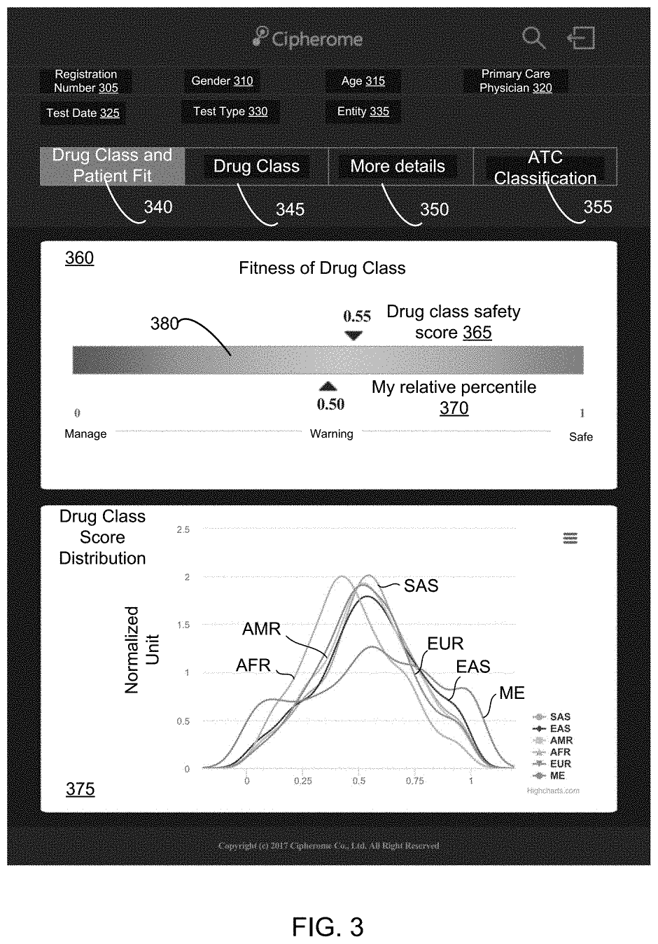

Each of FIGS. 3-17 depicts a UI that shows patient specific treatment information. In various embodiments, any one of the UIs shown in FIGS. 3-17 can be displayed in response to a selection of a patient (e.g., selection of a patient or patient information in the array 255). Additionally, as described in further detail below, the UIs of FIGS. 3-17 can be transitioned between one another.

FIG. 3 is an example UI that depicts treatment information for a patient corresponding to one drug class, in accordance with an embodiment. The example UI can include various background information 305-335 related to the patient and a conducted test. For example, the background information can include the registration number 305 of the patient, the gender 310 of the patient, the age 315 of the patient, the primary care physician 320 of the patient. Furthermore, the background information can include information related to conducted test such as when the test was conducted (e.g., test date 325), the type of test conducted (e.g., test type 330), and the entity 335 that conducted the test. In various embodiments, a type of test can refer to a type of disease that the test was conducted for. In some embodiments, the type of test can refer to the procedure of the test (e.g., blood draw, saliva swab, urine collection, and the like). In some embodiments, the test type refers to a sequencing test that is used to determine the genetic sequencing information for the patient.

As shown in FIG. 3, the example UI can further include one or more selectable tabs 340, 345, 350 or 355, each of which provides additional treatment information in response to being selected. Although four selectable tabs are shown in FIG. 3 (e.g., drug class and patient fit 340, drug class 345, more details 350, and ATC classification 355), in various embodiments additional or fewer tabs can be shown in the UI. Here, FIG. 3 depicts that the drug class and patient fit 340 tab is selected. For example, a selected tab can be delineated from the other non-selected tabs through one of a different tab fill color, tab shading, tab sizing, textual color, tab shape, textual font, textual size, and the like.

The example UI corresponding to the selected drug class and patient fit 340 tab can include one or more panels that each depicts a graph of the safety of a drug class for the patient. For example, a first panel 360 describes the fitness of drug class field for the patient. The first panel 360 includes a colored graph 380 that represents the safety of the drug class for the patient. For example, the colored graph 380 may be a gradient of shadings that changes across a color spectrum when moving from one end of the colored graph 380 to the other end. As shown in FIG. 3, one end of the colored graph 380 is colored in red whereas a second end of the colored graph 380 is colored green. The colored graph 380 may correspond to a continuous range of drug class scores as well as discrete classifications, such that it maps a range of drug safety scores or drug class scores to a range of colors. For example, the first end (e.g., in red) of the colored graph 380 corresponds to a minimum drug class score (score=0) with a discrete classification of “Manage.” The second end (e.g., in green) of the colored graph 380 corresponds to a maximum drug class score (score=1.0) with a discrete classification of “Safe.” Additionally, the middle of the colored graph 380 (e.g, in yellow) corresponds to a drug class score of 0.5 with a classification of “Warning.”

In various embodiments, the colored graph 380 is further depicted in the UI along with a drug class score 365, which is a patient-specific score that represents the average safety for the patient if administered a drug that is categorized in the drug class. The drug class score 365 may be an average of the drug scores of each drug in the drug class. As shown in FIG. 3, this particular drug class has a drug class score 365 of 0.55, which is located above the colored graph 380.

In various embodiments, the colored graph 380 is further depicted in the UI along with a relative percentile 370. As shown in FIG. 3, the relative percentile 370 for this drug class is 0.50, which is located below the colored graph 380. The relative percentile 370 represents the percentile of the drug class score 365 for the patient in relation to similar patients. As one example, similar patients may be patients from the same ethnic background, cultural background, country of origin, and the like.

The example UI can include a second panel 375 which is a drug class score distribution 375 graph. The second panel 375 depicts distributions of drug class scores for one or more patient groups, each patient group including patients of a common ethnic background, cultural background, country of origin, and the like. FIG. 3 specifically depicts distributions of South Asian (SAS) populations, East Asian (EAS) populations, Ad Mixed American (AMR), African (AFR), European (EUR), and Middle Eastern (ME). As shown in FIG. 3, the X-axis of the drug class score distribution 375 can be the range of drug class scores that matches the range of drug class scores that corresponds to the colored graph 380. The Y-axis of the drug class score distribution 375 can be a normalized unit that enables the comparison of drug class scores across the different patient groups. Each distribution corresponding to a patient group may be differently indicated (e.g., different color, different line thickness, different line style) in the graph to differentiate from a distribution corresponding to a different patient group.

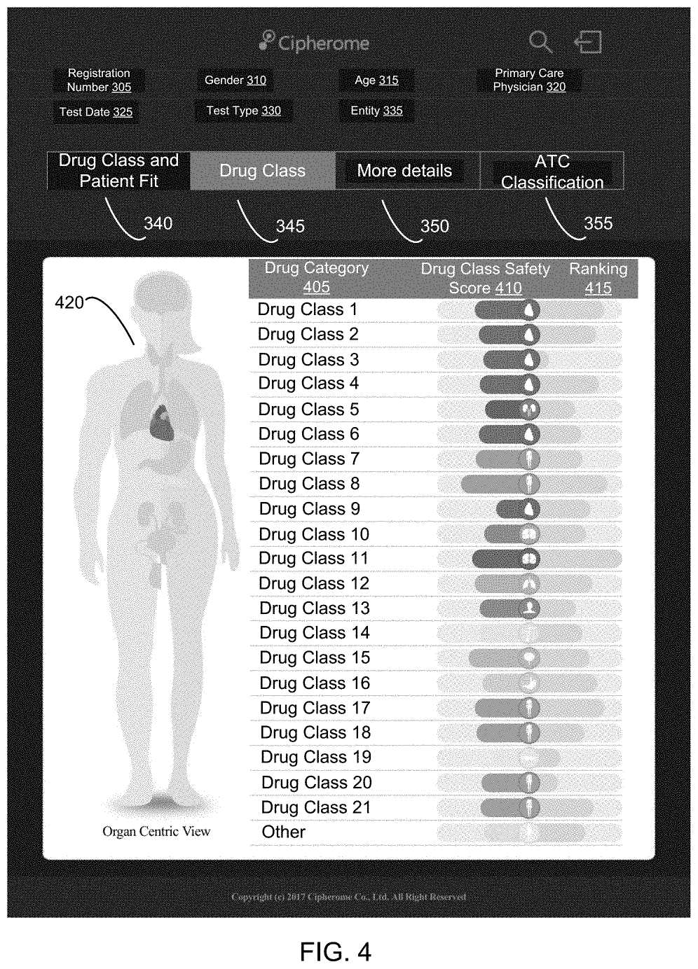

FIG. 4 is an example UI that depicts treatment information for a patient corresponding to multiple drug classes, in accordance with an embodiment. In various embodiments, the example UI shown in FIG. 4 is depicted responsive to a selection of the drug class 345 selectable tab. This example UI can also include the background information 305-335 as well as the selectable tabs 340, 345, 350, 355 as was described earlier in FIG. 3. Here, the drug class 345 tab is differently depicted to differentiate from the other, non-selected tabs 340, 350, and 355.

Generally, FIG. 4 is an example UI that depicts drug class scores 410 across different drug classes. Specifically, the example UI can include an anatomical depiction 420 of a human body, a column of drug categories 405 that includes the various drug classes, a column of drug class scores 410, where each score corresponds to a drug class, and optionally a ranking of each drug class in comparison to the other drug classes. In various embodiments, the ranking of each drug class is based on their respective drug class score 410 (e.g., highest rank is the drug class with the highest drug class score).

Referring to the anatomical depiction 420 of the human body, it may specifically show one or more organs of the body. Examples can include the brain, lungs, pancreas, heart, thyroid, liver, kidney, testes, and others. As shown in FIG. 4, one or more of the organs in the anatomical depiction 420 may be differently colored respective to other organs in the anatomical depiction 420. For example, the heart is shown in a first color (e.g., red) whereas the pancreas is shown in a second color (e.g., yellow).

Referring to the column of drug categories 405, FIG. 4 depicts a total of 21 different drug classes as well as an “other” category. In various embodiments, additional or fewer drug classes can be shown. Examples of drug classes include, but are not limited to: statins, lipid regulating agents, anti-coagulation agents, anti-arrhythmic agents, diuretics, angiotensin converting enzyme (ACE) inhibitors, calcium channel blockers, anti-depressants, anesthetics, respiratory agents, anti-allergy drugs, hormone regulating agents, steroids, digestive-regulating agents, painkillers, nonsteroidal anti-inflammatory drugs (NSAIDs), endocrine drugs, antibiotics, antifungals, antivirals, and others.

The drug class score 410 column includes a graphical depiction of each drug class score for a drug class and further includes a graphical depiction of the anatomical organ(s) that are impacted by drugs in the drug class. As shown in FIG. 4, each graphical depiction of a drug class score can be color-coded such that the color of the graphical depiction of the drug class score matches the color of an organ in the anatomical depiction 420, where the organ in the anatomical depiction 420 is impacted by the drugs in the corresponding drug class. For example, drug classes 1-4 each affect the heart. Therefore, the drug class score graph corresponding to each of drug classes 1-4 are color-coded in red which matches the color of the heart in the anatomical depiction 420. Thus, the example UI in FIG. 4 may include one or more interactive elements each having a graphical bar with a length proportion to a combination of one or more drug safety scores (i.e., drug safety scores for drugs in a respective drug class).

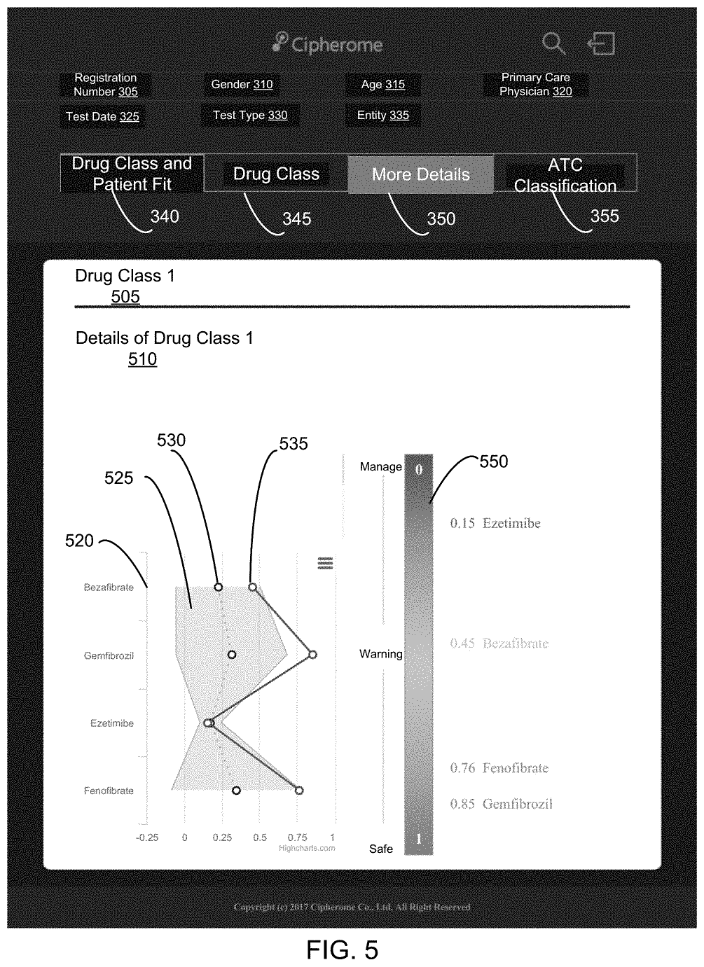

FIG. 5 is an example UI that depicts additional details for drugs of a drug class, in accordance with an embodiment. In various embodiments, the example UI shown in FIG. 5 is depicted responsive to a selection of the more details 350 selectable tab. In some embodiments, the example UI shown in FIG. 5 is depicted responsive to a selection of a drug class (e.g., drug class 1) located in the drug category 405 column (see FIG. 4). This example UI can also include the background information 305-335 as well as the selectable tabs 340, 345, 350, 355 as was described earlier in FIG. 3. Here, the more details 350 tab is differently depicted to differentiate from the other, non-selected tabs 340, 345, and 355.

Generally, FIG. 5 depicts an example UI that provides additional treatment information for the patient related to a drug class as well as individual drugs of the drug class. For example, the UI includes an identification of drug class 1 (505) as well as details of drug class 1 (510). The details of drug class 1 (510) can be textual information that describes various information such as the expected effects of drugs in drug class 1, the mode of action of drugs in drug class 1, organs in the body that are affected by drugs in drug class 1, possible side effects of drugs in drug class 1, and other information.

The example UI shown in FIG. 5 further includes one or more graphs that describe drug-specific treatment information for the patient. For example, FIG. 5 depicts a patient-drug score graph 520 and a colored graph 550. Referring first to the patient-drug score graph 520, the Y-axis of the graph 520 corresponds to specific drugs of drug class 1 (505) whereas the X-axis of the graph 520 refers to drug scores corresponding to each drug on the Y-axis. The patient-drug score graph 520 illustrates the drug scores for the patient in relation to drug scores for other patients in a common patient group. For example, as shown in FIG. 5, the patient-drug score graph 520 includes a first line 535 (e.g., red line) that corresponds to drug scores for the patient as well as a second line 530 (e.g., blue dotted line 530) that corresponds to average drug scores for patients in the common patient group (e.g., common ethnic background, cultural background, country of origin, and the like). Additionally, the patient-drug score graph 520 includes a deviation region 525 (e.g., shaded blue area 525) that represents the range of deviation of drug scores for patients in the common patient group. As an example, the deviation 525 represents a range of drug scores within one standard deviation from the average drug score for patients in the common patient group. Altogether, the patient-drug score graph 520 enables a user of the client device 110 to quickly understand treatment information for a patient pertaining to a drug or class of drugs in relation to other similar patients.

Referring now to the colored graph 550, it corresponds to a range of drug scores for drugs of drug class 1 (505). In various embodiments, the colored graph 550 is similarly configured to the colored graph 380 depicted in relation to FIG. 3. For example, one end (i.e., colored in red) of the colored graph 550 corresponds to a minimum drug score while the second end (i.e., colored in green) of the colored graph 550 corresponds to a maximum drug score. Additionally, each drug in drug class 1 (505) can be correspondingly displayed along the colored graph 550 based on the drug score associated with the drug. For example, Ezetimibe is associated with a drug score of 0.15 and therefore is displayed near the first end (e.g., red end) along the colored graph 550. Additionally, the text of the drug (e.g., Ezetimibe) and drug score (e.g., 0.15) may be displayed in a color that matches the color at the corresponding location of the colored graph 550. In various embodiments, the text of the drug and/or the drug score can be selectable such that when selected, additional details of the drug is displayed.

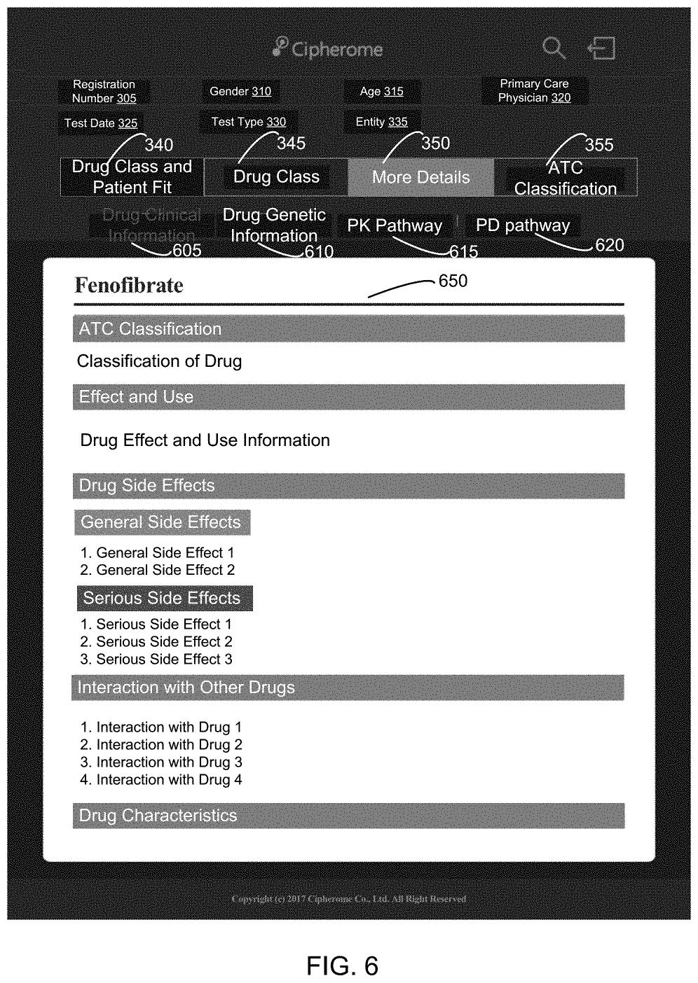

FIG. 6 is an example UI that depicts clinical details for a drug of a drug class, in accordance with an embodiment. In various embodiments, the example UI of FIG. 6 is depicted responsive to a selection of a drug in the drug class 1 (505) as shown in FIG. 5. The specific example UI shown in FIG. 6 can be depicted in response to a selection of the drug Fenofibrate (see FIG. 5) which is associated with a drug score of 0.76 and is located along the colored graph 550 according to its drug score. This example UI can similarly include the background information 305-335 as well as the selectable tabs 340, 345, 350, 355 as was described earlier in FIG. 3. Additionally, the example UI can include drug-specific selectable options 605, 610, 615, and 620 that each provide additional details for the drug of the drug class. As shown in FIG. 6, the drug-specific selectable options refer to drug clinical information 605, drug genetic information 610, pharmacokinetics (PK) pathway 615 related to the drug, and pharmacodynamics (PD) pathway 620 related to the drug. Here, the example UI illustrates clinical information of the drug and therefore, the drug clinical information 605 selectable option is differently displayed (e.g., red text) to differentiate from the other drug-specific selectable options.

The clinical information shown in FIG. 6 for the Fenofibrate drug are merely representative and is not meant to limit the type of clinical information displayed in an example UI. Examples of clinical information for the drug can include an ATC classification, drug effects and use information, general and serious side effects, predicted interactions with other drugs, drug characteristics, and references from where the clinical information was curated. The clinical information is located on a field 650 in the example UI. In one embodiment, the field 650 in the example UI is a scrollable field. Therefore, a first subset of clinical information can be displayed in the field 650. Responsive to receiving a user interaction (e.g., an upward scroll) on the field 650, a second subset of clinical information can be displayed in the field 650.

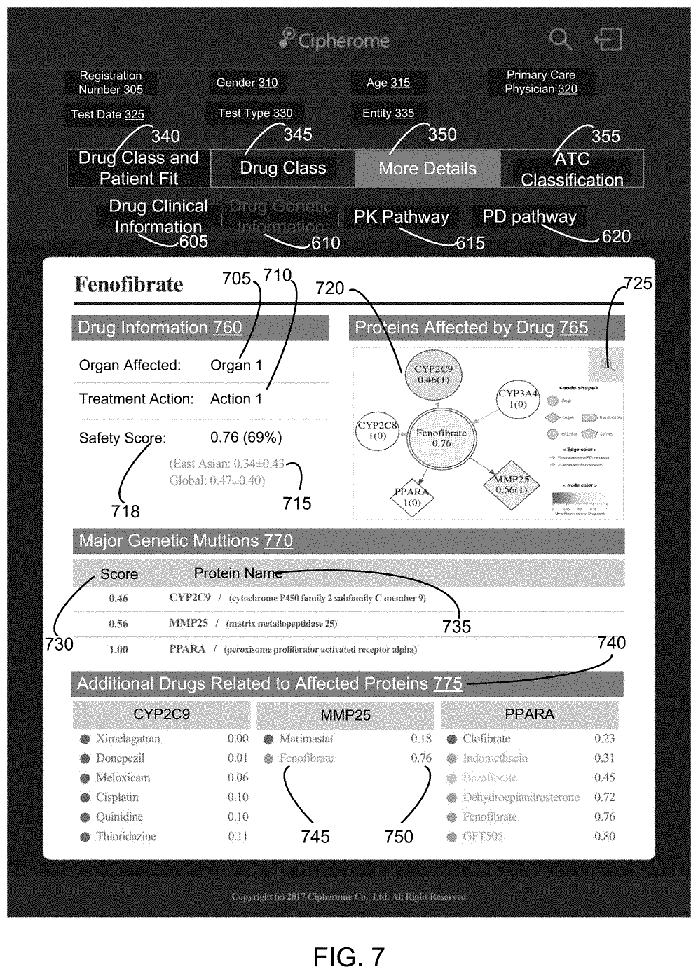

FIG. 7 is an example UI that depicts genetic information related to one drug in a drug class, in accordance with an embodiment. This example UI can similarly include the background information 305-335, the selectable tabs 340, 345, 350, 355, as well as the drug-specific selectable options 605, 610, 615, and 620 that each provide additional details for the drug of the drug class. Here in FIG. 7, the drug genetic information 610 selectable option is differently displayed (e.g., red text) to differentiate from the other drug-specific selectable options.

FIG. 7 depicts genetic information corresponding to proteins that are affected by a specific drug (fenofibrate). The example UI includes various panels such as a drug information 760 panel, a proteins affected by drug 765 panel, a major genetic mutations 770 panel, and additional drugs related to affected proteins 775 panel. The drug information 760 panel can depict organ information 705 that specifies the anatomical organ that the drug impacts. The drug information 760 panel further includes action information 710 that describes the mode of action of the drug. The drug information 760 panel further includes a safety score 718 for the drug. As shown in FIG. 7, the drug information 760 panel can include drug scores 715 for a patient group that the patient is a member of (e.g., East Asian) as well as all patients (e.g., global).

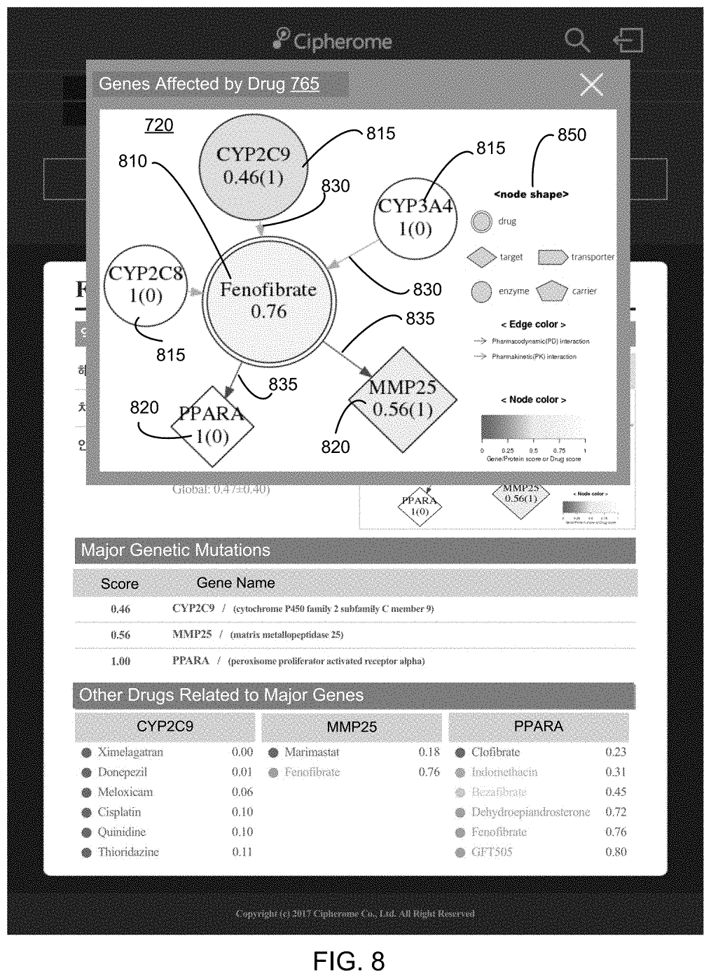

Referring to the panel depicting the proteins affected by drug 765, it includes a graphical depiction 720 of the drug as well as proteins and/or genes that are affected along the PK and PD pathways of the drug. The panel depicting the proteins affected by drug 765 can include a selectable option 725 such as a zoom-in option. The proteins affected by drug 765 panel is described in further detail below in relation to FIG. 8.

Referring to the major genetic mutations 770 panel, it includes a first column 730 of protein damage scores and a second column 735 that identifies proteins (such as transporter proteins, carrier proteins, and/or enzymes) that each correspond to a protein damage score in the first column 730. Each protein in the second column 735 (e.g., CYP2C9, MMP25, or PPARA) is involved in one of the PK or PD pathway of the drug (fenofibrate). In various embodiments, each protein listed in this major genetic mutations 770 panel is derived (e.g., transcribed and translated) from a gene sequence from the patient that may have one or more mutations.

Referring to the additional drugs related to affected proteins 775 panel, it displays multiple subpanels, each subpanel corresponding to one of the proteins listed in the second column 735 of the major genetic mutations 770 panel. For example, FIG. 7 depicts a first subpanel for the protein CYP2CP, a second panel for protein MMP25, and a third panel for protein PPARA. In each subpanel, one or more additional drugs 745 is depicted in conjunction with the drug score 750 for the additional drug. Each of the one or more additional drugs 745 shown in the subpanel impacts the protein of the subpanel. In various embodiments, the text that identifies each additional drug is depicted in a color that matches a color of the colored graph 550 (see FIG. 5). As one example, Ximelagatran is a drug that also impacts the CYP2C9 protein and is associated with a drug score of 0.00. Therefore, the text identifying the Ximelagatran drug is colored red to match the color of the minimal score on the colored graph 550.

FIG. 8 is an example UI that depicts an enlarged view of the panel depicting the proteins affected by a drug 765 information, in accordance with an embodiment. In various embodiments, the enlarged proteins affected by drug panel 765 is overlaid on top of the example UI depicted in FIG. 7. In some embodiments, the enlarged panel shown in FIG. 8 is displayed in response to an interaction with the selectable option 725 (see FIG. 7).

The enlarged proteins affected by drug panel 765 includes the graphical depiction 720 as well as a corresponding legend 850 that describes details of the graphical depiction 720. In some embodiments, the graphical depiction 720 is a directed graph that includes nodes and directional edges that connect each of the nodes.

The graphical depiction 720 illustrates the drug (e.g., fenofibrate) located within a first node 810 (e.g., a double circle). In various embodiments, the drug within the first node 810 is located at the center of the graphical depiction 720. In some embodiments, the drug within the first node 810 is the most prominently displayed element in the graphical depiction 720 to differentiate it beyond the other elements of the graphical depiction 720. Additionally, the graphical depiction 720 can include additional graphical elements (i.e., interactive elements) such as additional node 815 and 820. Each of the additional nodes 815 and 820 describe one of a target protein, enzyme, transporter protein, carrier protein, or other molecule that interacts with the drug (or metabolized compounds of the drug) through either a pharmacokinetic interaction or pharmacodynamics interaction. In various embodiments, the first node 810 and the additional nodes 815 and 820 differ in one of shape, size, shading, text color, and the like. Specifically, FIG. 8 depicts the first node 810 as a double wall circle, though other shapes can be used. The additional nodes 815 can be a shape different from the first node 810 such as one of a circle (single wall circle), polygon (e.g., triangle, quadrilateral, pentagon, hexagon), and the like.

In various embodiments, each of the first node 810 and additional nodes 815 and 820 can further include a score corresponding to the drug or protein of the node. As shown in FIG. 8, the drug of interest, fenofibrate, is displayed with a drug score of 0.76 whereas each of the proteins in the additional nodes 815 and 820 are displayed with a corresponding protein damage score. In various embodiments, each node 810, 815, and 820 can further be shaded with different colors based on the associated score. As shown in FIG. 8, the proteins with a protein damage score of 1 do not have shading (e.g., are in white) whereas CYP2C9 with a protein damage score of 0.46 and MMP25 with a protein damage score of 0.56 are different shades of yellow.

As shown in FIG. 8, the graphical depiction 720 further includes edges 830 and 835 that each connect the first node 810 to one of the additional nodes 815 or 820. In various embodiments, the edges 830 and 835 are directional. For example, edge 830 refers to a directional edge from an additional node 815 or 820 to the first node 810. In contrast, edge 835 refers to a directional edge from the first node 810 to the additional node 815 or 820. As shown in FIG. 8, each edge 830 may be depicted in a first color whereas each edge 835 may be depicted in a second color.

Referring now to the legend 850 in the proteins affected by drug 765 panel, it describes the nodes 810, 815, and 820, edges 830 and 835, as well as further information corresponding to either nodes 810, 815, and 820 or edges 830 and 835. For example, a first portion of the legend 850 depicts each of the shapes corresponding to the first node 810 and additional nodes 815 and 820 shown in the graphical depiction 720. Additionally, the legend 850 identifies the corresponding drug, target protein, transporter protein, enzyme, or carrier protein for the node 810, 815, and 820. A second portion of the legend 850 can describe the edges 830 and 835 shown in the graphical depiction 720. In particular, the second portion of the legend 850 depicts a representation, such as a colored representation, of each type of edge 830 and 835 as well as the type of interaction that each edge 830 and 835 corresponds to (e.g., PD interaction or PK interaction). A third portion of the legend 850 can depict a colored graph that corresponds to the colored shading of each node 810, 815, or 820. Specifically, the color of each node corresponds to the protein damage score or drug score of the node and is depicted along the x-axis of the colored graph of legend 850. Thus, the colored graph may map a range of protein damage scores or drug safety scores to a range of colors.

User Interface Depicting PK and PD Pathways

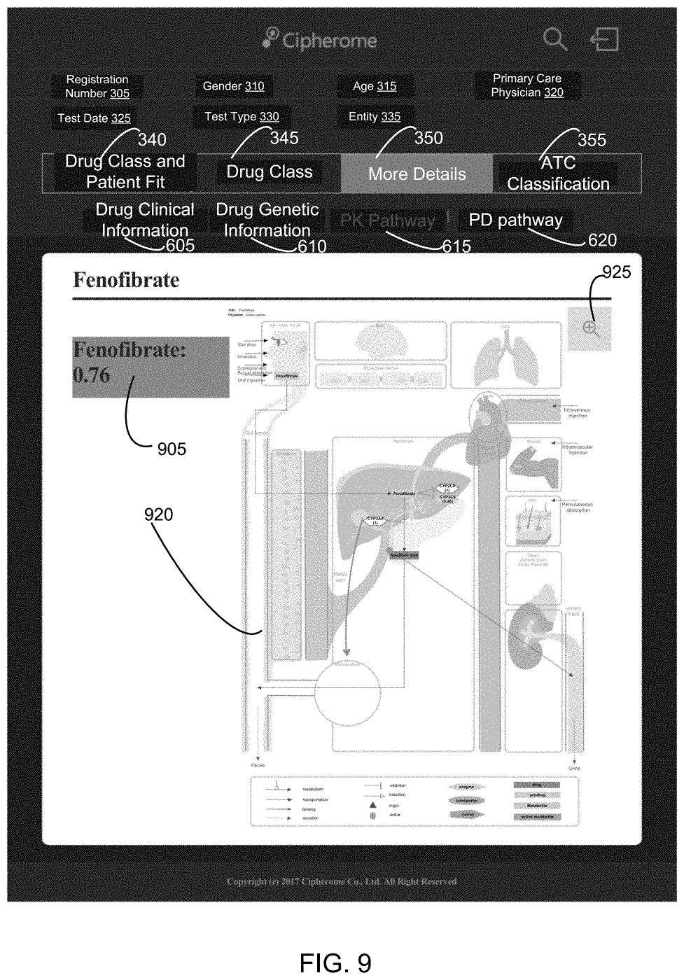

FIG. 9 is an example UI that depicts pharmacokinetic (PK) pathways for a drug, in accordance with an embodiment. This example UI can similarly include the background information 305-335, the selectable tabs 340, 345, 350, 355, as well as the drug-specific selectable options 605, 610, 615, and 620 that each provide additional details for the drug of the drug class. Here in FIG. 9, the PK pathway 615 selectable option is differently displayed (e.g., red text) to differentiate from the other drug-specific selectable options.

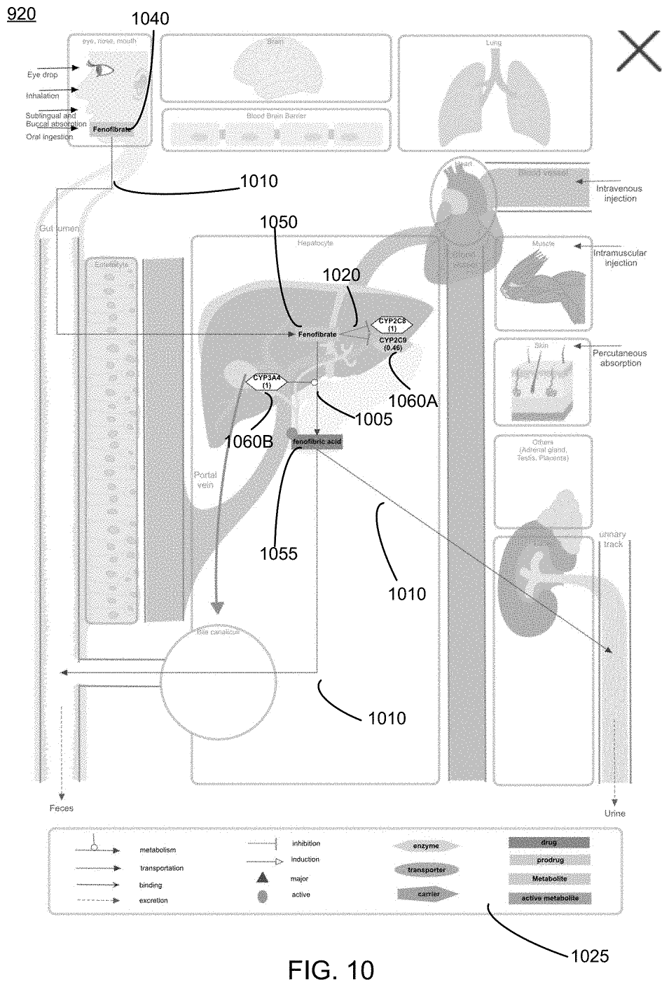

In various embodiments, the example UI depicting the PK pathways includes an identification of the drug (e.g., fenofibrate) and the drug score (e.g., 0.76) of the drug located in a first region 905 of the example UI. An additional region 920 of the example UI depicts the overall PK pathways that the drug is involved in. Further discussion of the depiction of the PK pathways is described below in relation to FIG. 10. The example UI can further include a selectable option 925 that, when interacted with, enlarges the additional region 920 that depicts the overall PK pathways.

FIG. 10 is an example UI that depicts an enlarged view of the PK pathways for the drug, in accordance with an embodiment. The example UI can include two portions: 1) a visual depiction of the PK pathways and 2) a legend 1025 that describes the various indications that are shown in relation to visual depiction of the PK pathways.

In various embodiments, the visual depiction of the PK pathways includes various depictions of anatomical organs such as the eye, nose, mouth, brain, blood brain barrier, lung, heart, muscle, skin, kidney, liver, adrenal glands, testis, intestines, and placenta. The example UI includes various depictions of transport structures such as blood vessels (e.g., arteries and veins), bile ducts, and excretory tracts (e.g., urinary tract) that serve to connect each of the depicted anatomical organs. The example UI includes various methods of administration that a drug can be provided to a patient such as eye drop, inhalation, sublingual and buccal absorption, oral ingestion, intravenous injection, intramuscular injection, and percutaneous absorption. Other anatomical organs, transport structures, and administration methods can be included in other embodiments.

The visual depiction of the PK pathways further includes the drug, one or more proteins that are involved in the PK pathway of the drug, as well metabolic pathways of the drug. The visual depiction may include a set of interactive elements representing proteins affected by the drugs or the drugs themselves or metabolites generated from the drugs. The example UI includes a first node 1040 that corresponds to the drug. The first node 1040 is overlaid on one or more of the anatomical organs and corresponds to one of the methods of administration. As shown in FIG. 10, the first node 1040 corresponds to the drug, fenofibrate, and is overlaid on top of a depiction of a patient's mouth. Additionally, the first node 1040 is located adjacent to the depiction of the oral ingestion method of administration, thereby indicating that the drug is orally administered to the patient. The example UI further includes a second node 1050 that also corresponds to the drug. The second node 1050 is overlaid with a depiction of an anatomical organ where the drug is metabolized. For example, as shown in FIG. 10, the second node 1050 corresponding to fenofibrate is overlaid with the liver, indicating that fenofibrate is metabolized within the liver.

Generally, the first node 1040 and the second node 1050 are connected by a directional edge 1010. This directional edge 1010 depicts the transportation of the drug from its method of administration to the anatomical organ where the drug is metabolized. As shown in FIG. 10, the directional edge 1010 travels from the location where the drug is administered (e.g., mouth), into the gut lumen, absorbed by enterocytes, through the portal vein, and into the liver.

The second node 1050 may further have one or more additional directional edges 1005 and 1020 that each describes an interaction encountered by the drug of the second node 1050. For example, each additional directional edge 1005 and 1020 can refer to one of a metabolism, transportation, binding, excretion, inhibition, or induction interaction. Additionally, each directional edge 1005 and 1020 can be depicted differently based on the type of pathway. For example, as shown in FIG. 10, an inhibition pathway is depicted as a red, blocked edge 1020 whereas the metabolic pathway is depicted as a green, directional arrow 1005.

In various embodiments, each additional directional edge 1005 and 1020 is associated with one or more additional nodes that are each impacted by the drug of the second node 1050. As one example, each inhibition pathway 1020 can be directed to an additional node 1060A that corresponds to a protein that is inhibited by the drug. Specifically, FIG. 10 depicts that fenofibrate inhibits CYP2C8 and CYP2C9. As another example, each metabolic pathway 1005 can be associated with an additional node 1060B that corresponds to a protein that metabolizes the drug. Specifically, FIG. 10 depicts that CYP3A4 metabolizes fenofibrate to fenofibric acid, which is the active form of the drug. Each additional node 1060 can further depict the protein damage score associated with each corresponding protein of that additional node 1060. Furthermore, each additional node 1060 can be shaded a color based on the protein damage score. The shaded color of the additional node 1060 can align with the colored graph shown in the legend 850 (see FIG. 8).

FIG. 10 further depicts a third node 1055 that represents the metabolites (fenofibric acid) that are metabolized from the initial drug (fenofibrate). Specifically, the example UI depicts the elimination of the metabolite (fenofibric acid) through one or more directional edges 1010 that represent the transportation of the metabolite. Here, fenofibric acid is transported through the bile canaliculi to be excreted through the feces and also transported through the blood, kidney, and excreted through the urinary track. Each of the nodes that correspond to a version of the drug (e.g., first node 1040, second node 1050, third node 1055) can be shaded to indicate the various versions of the drug. For example, versions of the drug can be one of the drug, prodrug, metabolite, and active metabolite.

In various embodiments, the example UI is an interactive UI. For example, each node 1040, 1050, 1055, 1060 depicted in the example UI is selectable to display additional information for the prodrug, drug, metabolized drug, and protein that each node corresponds to.

Referring now to the legend 1025, it illustrates the various depictions of nodes, directional edges, version of drug, and the like. For example, as shown in FIG. 10, the legend 1025 illustrates the different types of pathways (e.g., metabolism, transportation, binding, excretion, inhibition, and induction) that are each represented by a directional edge that differs from other directional edges based on one of color, line style, arrow head style, arrow fill, and the like. The legend 1025 includes indications as to whether a node corresponding to a version of the drug is one of a major or active form (see purple triangle and red oval). The legend 1025 includes indications of the type of protein (e.g., enzyme, transporter, carrier) that an additional node 1060 corresponds to. Such an indication can be uniquely embodied based on a shape of the node and/or a color fill of the node. The legend 1025 includes indications of the version of a drug that a node (such as first node 1040, second node 1050, and third node 1055) correspond to. Here, each indication of a version of a drug can be uniquely embodied based on a shape of the node and/or a color fill of the node.

FIG. 10 displays an interactive UI that depicts protein damage scores for proteins that are affected by the administered drug. In various embodiments, the interactive UI can further include the drug score (e.g., drug score for fenofibrate or fenofibric acid in the corresponding node 1040, 1050, or 1055). In some embodiments, the interactive UI only includes the drug score without the protein damage scores.

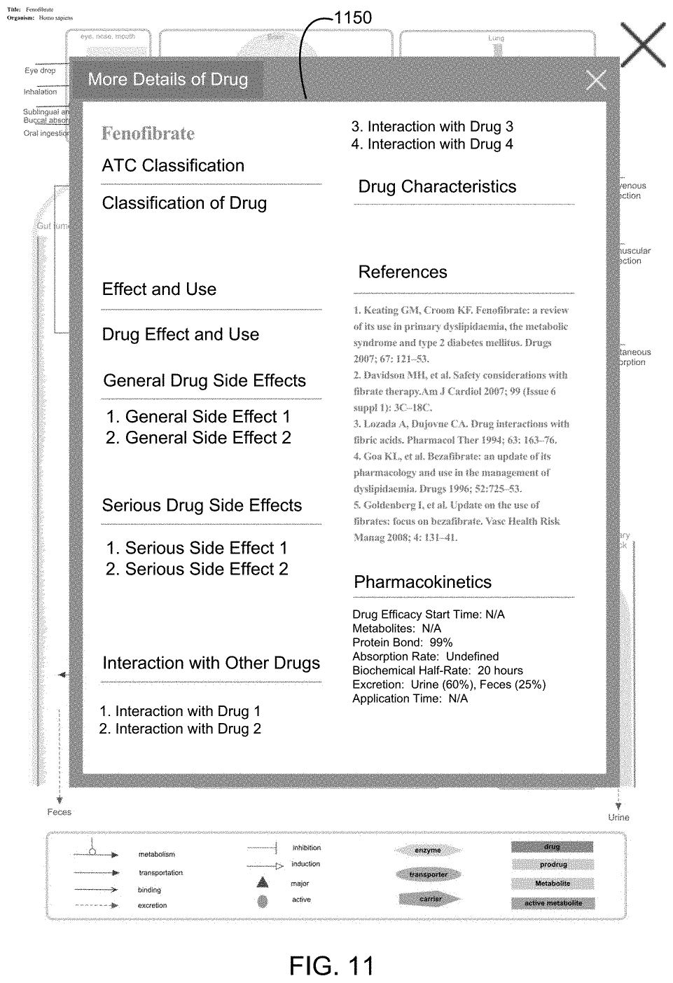

FIG. 11 is an example UI that depicts additional details of a drug overlaid with the PK pathways for the drug, in accordance with an embodiment. In various embodiments, the example UI in FIG. 11 is depicted in response to a user selection with one of the drug related nodes (e.g., first node 1040 or second node 1050) in the example UI shown in FIG. 10.

The example UI in FIG. 11 includes an informational drug panel 1150 that depicts additional details of the drug, such as fenofibrate. In various embodiments, the additional details of the drug include an ATC classification of the drug, effects and use of the drug, general and serious side effects of the drug, interaction with other drugs, drug characteristics, references, and additional information. In various embodiments, such additional details may be similar to or may match the clinical drug information shown in the field 650 (see FIG. 6).

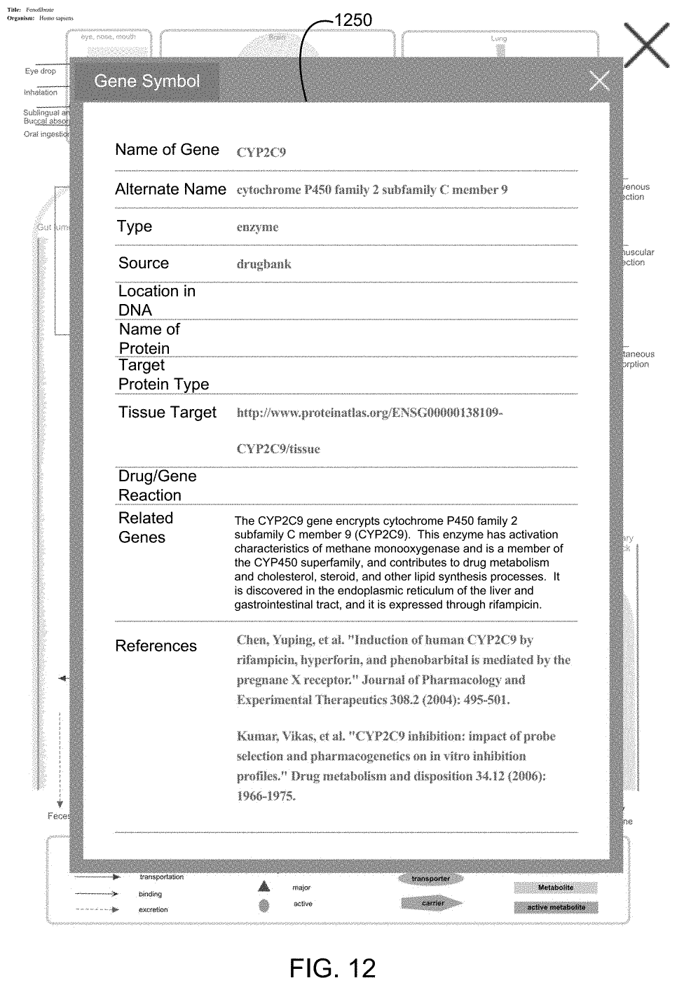

FIG. 12 is an example UI that depicts additional details for a gene overlaid with the PK pathways for the drug, in accordance with an embodiment. In various embodiments, the example UI in FIG. 12 is depicted in response to a user selection with one of the protein related nodes (e.g., additional nodes 1060 and more specifically, additional node 1060A corresponding to CYP2C9) in the example UI shown in FIG. 10.

Additional details of a gene can include the name of the gene, alternate name of the gene, type, source of the additional details of the gene, location in the DNA that the gene is located, name of protein translated from the gene, the type of the protein that is the target, the tissue target, drug/gene reactions, related genes, and references.

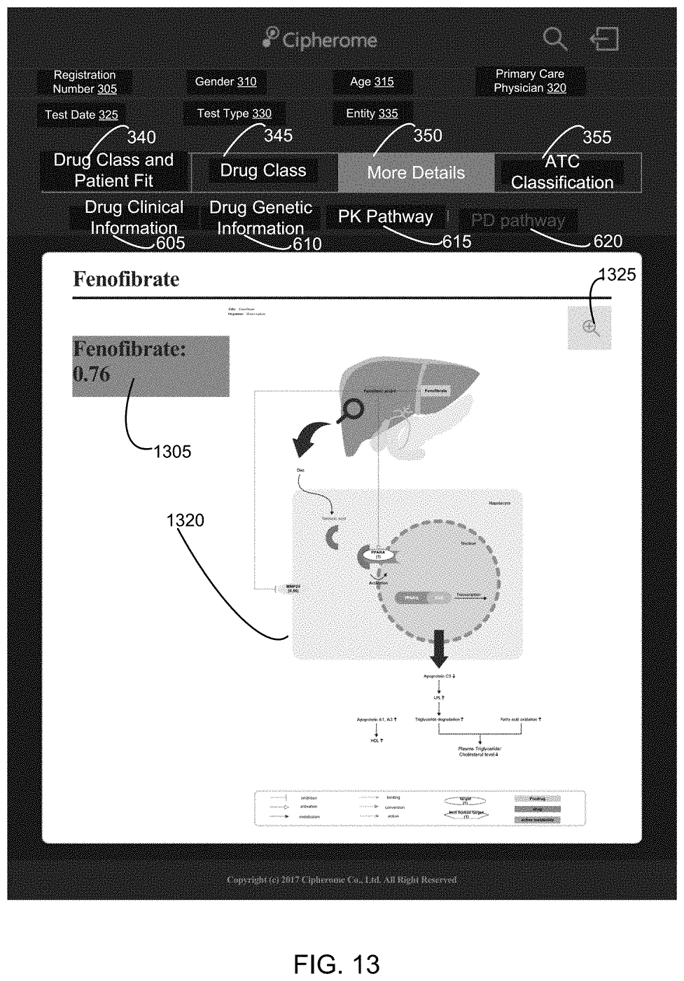

FIG. 13 is an example UI that depicts PD pathways for a drug, in accordance with an embodiment. This example UI can similarly include the background information 305-335, the selectable tabs 340, 345, 350, 355, as well as the drug-specific selectable options 605, 610, 615, and 620 that each provide additional details for the drug of the drug class. Here in FIG. 13, the PD pathway 620 selectable option is differently displayed (e.g., red text) to differentiate from the other drug-specific selectable options.

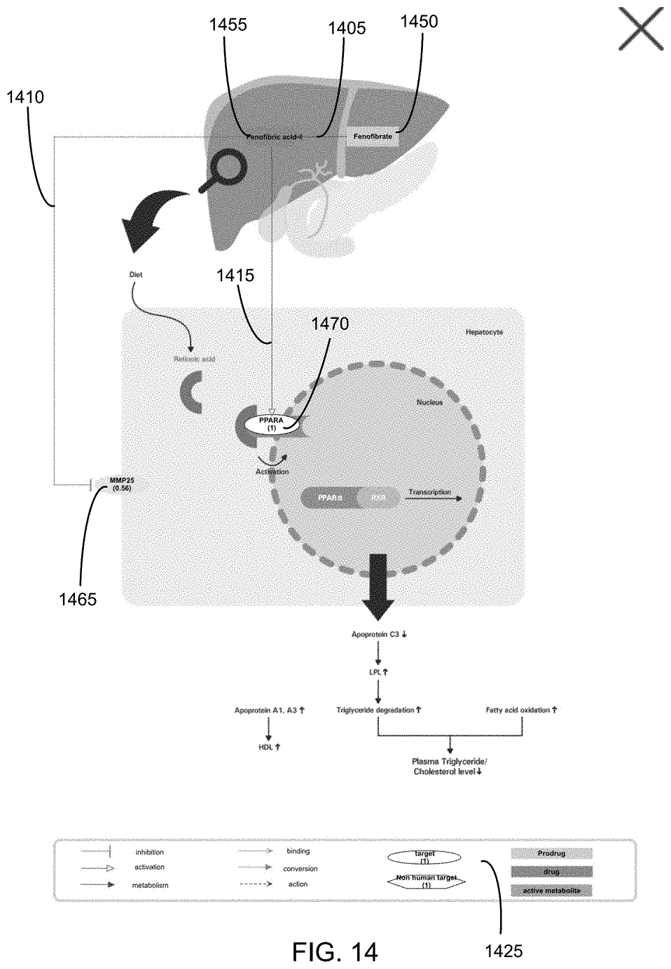

In various embodiments, the example UI depicting the PD pathways includes an identification of the drug (e.g., fenofibrate) and the drug score (e.g., 0.76) of the drug located in a first region 1305 of the example UI. An additional region 1320 of the example UI depicts the overall PD pathways that the drug is involved in. Further discussion of the depiction of the PD pathways is described below in relation to FIG. 14. The example UI can further include a selectable option 1325 that, when interacted with, enlarges the additional region 1320 that depicts the overall PD pathways.

FIG. 14 is an example UI that depicts an enlarged view of PD pathways for the drug, in accordance with an embodiment. Similar to the example UI depicting the PK pathways (see FIG. 10), the example UI depicting the PD pathways here also includes various nodes and directional edges that depict the interactions of the drug.

The example UI can include two portions: 1) a visual depiction of the PD pathways and 2) a legend 1425 that describes the various indications that are shown in relation to visual depiction of the PD pathways.

In various embodiments, the visual depiction of the PD pathways includes depictions of one or more anatomical organs that the drug interacts with such as the eye, nose, mouth, brain, blood brain barrier, lung, heart, muscle, skin, kidney, liver, adrenal glands, testis, intestines, and placenta. Specifically, FIG. 14 depicts that the drug (fenofibrate) interacts with the liver. Additionally, the visual depiction of the PD pathways can include a cellular-level depiction of the anatomical organ that the drug interacts with. FIG. 14 depicts a single hepatocyte as well as one or more cell organelles of the hepatocyte including any of the nucleus, endoplasmic reticulum, mitochondria, golgi apparatus, lysosomes, peroxisomes, vesicles, cell membrane, ribosomes, and the like.

The depiction of PD pathways further includes the drug and one or more proteins that are involved in the PD pathway. The example UI includes a first node 1450 that corresponds to the administered drug and a second node 1455 that corresponds to the metabolized version of the drug. Each of the first node 1450 and second node 1455 are overlaid with the anatomical organ, such as the liver, that interacts with the drug. The first node 1450 and the second node 1455 are connected by a directional edge 1415 that indicates the metabolism of the administered drug to metabolites.

The second node 1455 is connected through one or more additional edges 1410 and 1415 to one or more additional nodes 1465 and 1470 that each corresponds to a protein. Each additional edge 1410 can represent one of an inhibition, activation, binding, metabolism, conversion, or action. Each of the additional nodes 1465 and 1470 is overlaid with the cellular-level depiction of the anatomical organ. Additionally, each additional node 1465 and 1470 is located at the cellular organelle that the corresponding protein is located when it interacts with the drug of the second node 1455. As an example, FIG. 14 specifically depicts an additional node 1465 corresponding to the MMP25 protein which is associated with the plasma membrane of the cell. Additionally, FIG. 14 depicts an additional node 1470 corresponding to the Peroxisome Proliferator Activator Receptor Alpha (PPARA) protein that is associated with the nuclear membrane of the nucleus. Each additional node 1465 and 1470 includes a protein damage score of the protein corresponding to the additional node 1465 and 1470. In various embodiments, the shading of each additional node 1465 and 1470 is based on the protein damage score that aligns with the x-axis of the colored graph of legend 850 (see FIG. 8).

In various embodiments, supplementary proteins and/or molecules that are involved in the PD pathway are also visually depicted in conjunction with the cellular-level depiction of the anatomical organ. Supplementary proteins and/or molecules may be molecules that do not directly interact with the drug (or prodrug or drug metabolite), but can interact directly or indirectly with a protein involved the PD pathway of the drug. For example, FIG. 14 depicts the presence of the supplementary protein (retinoic acid) that additionally binds the PPARA protein to facilitate the activation of PPARA when it interacts with fenofibric acid.

In various embodiments the PD pathway further depicts the direct and indirect effects of the drug. For example, FIG. 14 depicts that the PPAR-alpha gene is impacted as well as the overall levels of apoprotein C3, lipoprotein lipase (LPL), triglyceride degradation, fatty acid oxidation, plasma triglyceride/cholesterol levels, apoprotein A1, apoprotein A3, and high-density lipoprotein (HDL).

Referring now to the legend 1425, it illustrates the various depictions of nodes, directional edges, version of drug, and the like. For example, as shown in FIG. 14, the legend 1425 illustrates the different types of pathways (e.g., inhibition, activation, metabolism, binding, conversion, and action) that are each represented by a directional edge that differs from other directional edges based on one of color, line style, arrow head style, arrow fill, and the like. The legend 1425 includes indications as to whether an additional node 1465 or 1470 corresponding to a protein is one of a human or non-human target. Such an indication can be uniquely embodied based on a shape of the node and/or a color fill of the node. The legend 1425 includes indications of the version of a drug that a node (such as first node 1450 or second node 1455) correspond to. Here, each indication of a version of a drug can be uniquely embodied based on a shape of the node and/or a color fill of the node.

In various embodiments, the example UI is an interactive UI. For example, each node 1450, 1455, 1465, 1470 depicted in the example UI is selectable to display additional information for the prodrug, drug, metabolized drug, and protein that each node corresponds to.

FIG. 14 displays an interactive UI that depicts protein damage scores for proteins that are affected by the administered drug. In various embodiments, the interactive UI can further include the drug score (e.g., drug score for fenofibrate or fenofibric acid). In some embodiments, the interactive UI only includes the drug score without the protein damage scores.

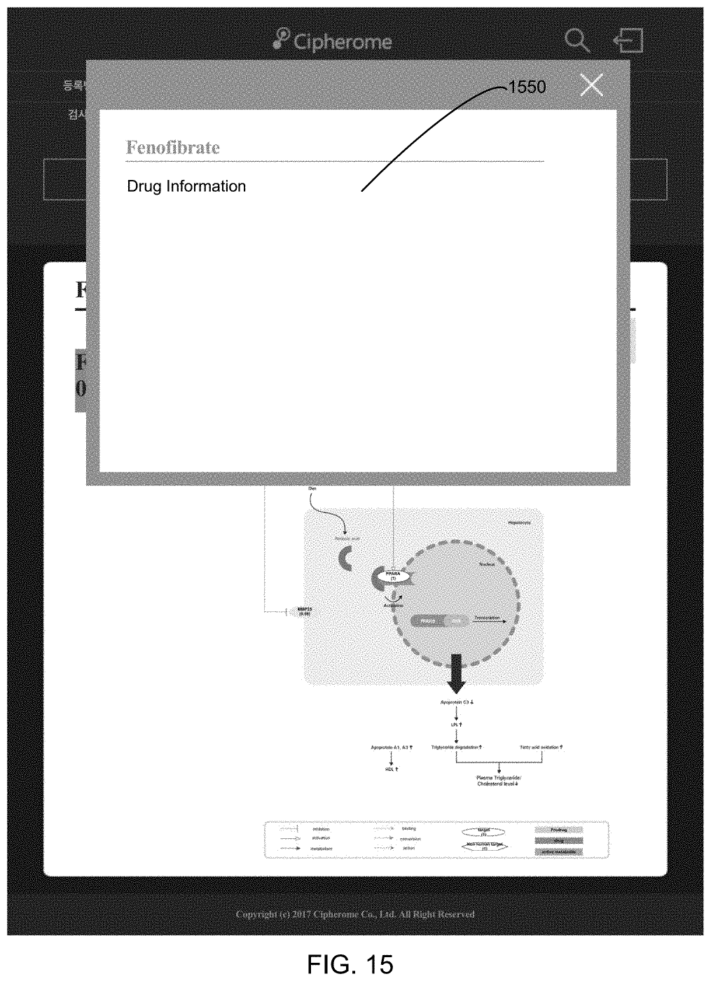

FIG. 15 is an example UI that depicts additional details of a drug overlaid with the PD pathways for the drug, in accordance with an embodiment. In various embodiments, the example UI in FIG. 15 is depicted in response to a user selection of the drug node (e.g., first node 1450) in the example UI shown in FIG. 14. The additional details of the drug can include any of the aforementioned additional details described in relation to FIG. 11.

User Interface Depicting Information for Different Classifications of Drugs

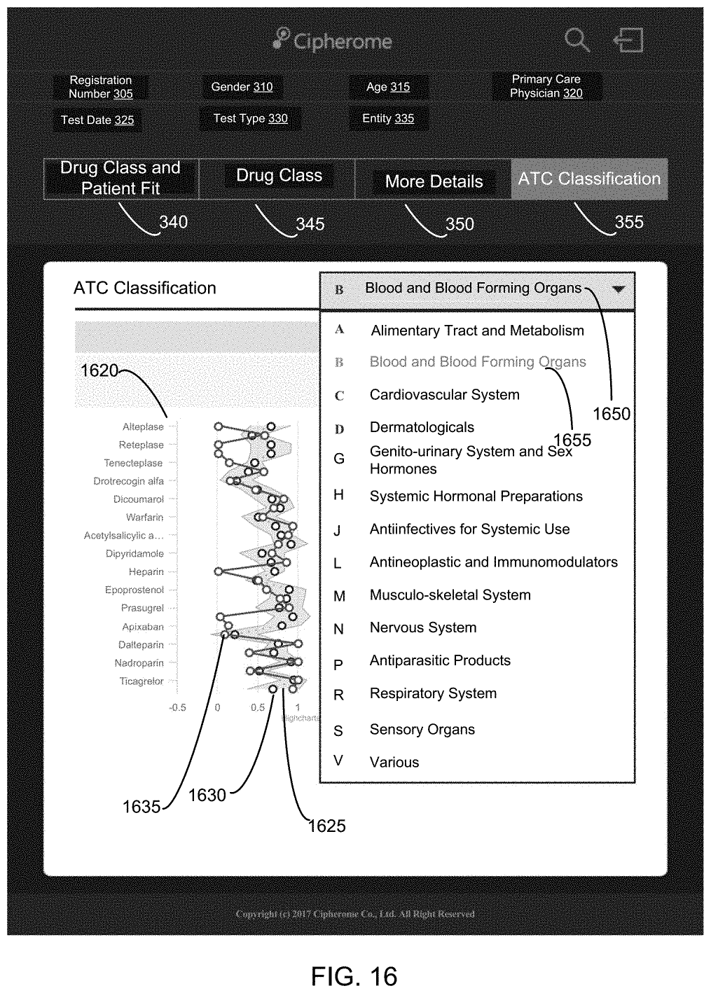

FIG. 16 is an example UI that depicts drug scores of varying drugs in a first category based on the anatomical therapeutic chemical (ATC) classification system, in accordance with an embodiment. This example UI can similarly include the background information 305-335 and the selectable tabs 340, 345, 350, 355. Here, the ATC classification 355 selectable tab is selected and is differently displayed in relation to the other selectable tabs 340, 345, and 350.

Generally, the example UI for the ATC classification selectable tab 355 depicts drug scores for multiple drugs that are in the same category, as classified by the ATC classification. For example, the drug scores can be depicted as a patient-drug score graph 1620, similar to the patient-drug score graph 520 previously described in relation to FIG. 5. The Y-axis of the graph 1620 refers to drugs of the drug category, as classified by the ATC classification. Specifically, the patient-drug score graph 1620 shown in FIG. 16 depicts drugs in category B (Blood and Blood forming Organs) of the ATC classification. The X-axis of the graph 1620 refers to drug scores corresponding to each drug on the Y-axis. The patient-drug score graph 1620 includes a first line 1635 (e.g., red line) that corresponds to drug scores for the patient and a second line 1630 (e.g., blue dotted line) that corresponds to average drug scores for patients in the common patient group (e.g., common ethnic background, cultural background, country of origin, and the like). Additionally, the patient-drug score graph 1620 includes a deviation 1625 (e.g., shaded blue area) that represents the range of deviation of drug scores for patients in the common patient group.

The example UI further includes a drop down menu 1650 that enables access to the variety of drugs from different ATC classification categories. As shown in FIG. 16, the currently selected category 1655 (e.g., category B) is indicated in the drop down menu as selected.

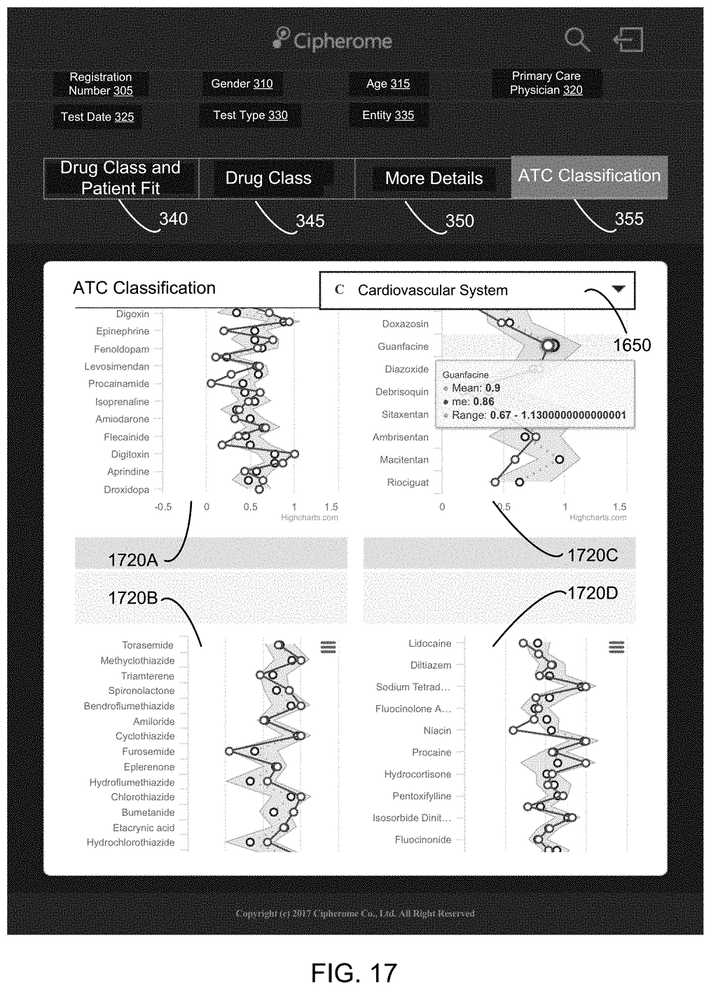

FIG. 17 is an example UI that depicts drug scores of various drugs in a second category based on the ATC classification system, in accordance with an embodiment. In various embodiments, the example UI of FIG. 17 is depicted in response to the selection of a different category (e.g., category C) from the drop-down menu 1650 shown in FIG. 16. Here, multiple patient-drug score graphs 1720A-D can be depicted in a scrollable interface. In one embodiment, additional drug scores of each patient-drug score graph 1720A-D can be displayed in response to a scrolling user interaction.

### Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

While the invention has been particularly shown and described with reference to various embodiments, it will be understood by persons skilled in the relevant art that various changes in form and details can be made therein without departing from the spirit and scope of the invention. All references, issued patents and patent applications cited within the body of the instant specification are hereby incorporated by reference in their entirety, for all purposes.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

A module may represent a functional or structural combination of hardware for implementing the technical spirit of the present invention and software for driving the hardware. For example, the module may be a predetermined code and a logical unit of a hardware resource by which the predetermined code is executed. A module does not necessarily mean physically connected codes or one kind of hardware.

The response prediction system described above can be implemented in hardware, firmware, software, or combinations thereof. If the system is implemented in software, a storage medium may include any storage or transmission medium readable by a device such as a computer. For example, the computer-readable medium may include a ROM (read only memory); a RAM (random access memory); a magnetic disc storage medium; an optical storage medium; a flash memory device; and other electric, optical or acoustic signal transmission medium.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A computer-implemented method for displaying treatment information for a patient for one or more drugs, the method comprising:
receiving individual gene sequence information of the patient;
obtaining treatment information including a set of drug safety scores for a set of drugs or a set of protein damage scores, wherein a protein damage score indicates a predicted dysfunction of a respective protein in the patient based on the gene sequence information of the patient, and wherein a drug safety score indicates a predicted adverse reaction to a respective drug for the patient based on the protein damage scores for proteins associated with pharmacokinetic (PK) or pharmacodynamic (PD) pathway of the respective drug;
receiving, from a client device, a request for an interactive user interface providing information on administering the one or more drugs to the patient;
generating instructions for display of a first screen of the interactive user interface, the first screen configured to display:
a visual depiction of a set of anatomical organs of a human body involved in PK or PD pathway of the one or more drugs, and
a set of interactive elements on the first screen, wherein each interactive element is displayed in association with a respective anatomical organ, and wherein the interactive element displays an indication of protein damage scores or drug safety scores based on the treatment information;
providing the instructions to the client device for display of the first screen of the interactive user interface.

2. The computer-implemented method of claim 1, wherein the treatment information includes at least the set of drug safety scores, and wherein each interactive element is a graphical bar having a length proportional to a combination of one or more drug safety scores for the one or more drugs.

3. The computer-implemented method of claim 2, wherein the set of anatomical organs are each assigned a respective color, and wherein each interactive element is displayed with a color assigned to the anatomial organ in association with the interactive element.

4. The computer-implemented method of claim 1, wherein the treatment information includes at least the set of protein damage scores, and wherein the set of interactive elements represent a set of proteins affected by the one or more drugs, and wherein each interactive element is overlaid on the anatomical organ where the PK or PD pathway involving the protein of the interactive element occur.

5. The computer-implemented method of claim 4, wherein each interactive element is displayed with the indication of a protein damage score for the protein represented by the interactive element.

6. The computer-implemented method of claim 5, further comprising generating a color graph mapping a range of protein damage scores to a range of colors, and wherein the indication for the interactive element is a respective color for the protein damage score for the interactive element obtained from the color graph.

7. The computer-implemented method of claim 1, wherein the set of anatomical organs involved in the PK or PD pathway are anatomical organs related to administration, transportation, or metabolism of the one or more drugs in the patient's body.

8. The computer-implemented method of claim 1, wherein the first screen further displays another set of interactive elements representing the one or more drugs or metabolites generated from the one or more drugs, wherein each interactive element is overlaid on the anatomical organ where the PK or PD pathway involving the one or more drugs of the interactive element occur.

9. The computer-implemented method of claim 8, wherein the first screen further comprises a directed edge from a first interactive element representing a drug overlaid on a first anatomical organ where the drug is administered to a second interactive element representing the drug overlaid on a second anatomical organ where the drug is transported or metabolized.

10. The computer-implemented method of claim 1, further comprising, responsive to receiving another request from the client device, generating a second screen of the interactive user interface, the second screen displaying:
a color graph mapping a range of drug safety scores to a range of colors, and
an indication marking the drug safety scores of the one or more drugs for the patient on the color graph.

11. The computer-implemented method of claim 1, wherein a protein damage score for a respective protein is computed from one or more genome sequence variation scores for one or more genome sequence variations in the patient, each genome sequence variation score indicating a degree of genome sequence variation in the patient that causes change to structure and/or function of the respective protein.

12. The computer-implemented method of claim 1, further comprising, responsive to receiving another request from the client device, generating a second screen of the interactive user interface, the second screen displaying:
a line graph mapping the drug safety scores of the one or more drugs for the patient, wherein the one or more drugs have a same classification under anatomical therapeutical classification (ATC) system.

13. The computer-implemented method of claim 1, wherein the first screen further displays a visual depiction of protein damage scores, drug safety scores or their distribution in a population obtained using gene sequence information of the population.

14. The computer-implemented method of claim 13, wherein the first screen further displays a visual representation of the protein damage scores or the drug safety scores of the patient, relative to the protein damage scores, drug safety scores, or their distribution in the population.

15. The computer-implemented method of claim 13, wherein the population represents an ethnic, racial, gender, or age group.

16. A system for displaying treatment information for a patient for one or more drugs, the system comprising:
a processor;
a computer readable storage medium for storing instructions executable by the processor, the instructions comprising:
receiving individual gene sequence information of the patient;
obtaining treatment information including a set of drug safety scores for a set of drugs or a set of protein damage scores, wherein a protein damage score indicates a predicted dysfunction of a respective protein in the patient based on the gene sequence information of the patient, and
wherein a drug safety score indicates a predicted adverse reaction to a respective drug for the patient based on the protein damage scores for proteins associated with pharmacokinetic (PK) or pharmacodynamic (PD) pathway of the respective drug;
receiving, from a client device, a request for an interactive user interface providing information on administering the one or more drugs to the patient;
generating instructions for display of a first screen of the interactive user interface, the first screen configured to display:
a visual depiction of a set of anatomical organs of a human body involved in PK or PD pathway of the one or more drugs, and
a set of interactive elements on the first screen, wherein each interactive element is displayed in association with a respective anatomical organ, and wherein the interactive element displays an indication of protein damage scores or drug safety scores based on the treatment information;
providing the instructions to the client device for display of the first screen of the interactive user interface.

17. The system of claim 16, wherein the treatment information includes at least the set of drug safety scores, and wherein each interactive element is a graphical bar having a length proportional to a combination of one or more drug safety scores for the one or more drugs.

18. The system of claim 17, wherein the set of anatomical organs are each assigned a respective color, and wherein each interactive element is displayed with a color assigned to the anatomial organ in association with the interactive element.

19. The system of claim 16, wherein the treatment information includes at least the set of protein damage scores, and wherein the set of interactive elements represent a set of proteins affected by the one or more drugs, and wherein each interactive element is overlaid on the anatomical organ where the PK or PD pathway involving the protein of the interactive element occur.

20. The system of claim 19, wherein each interactive element is displayed with the indication of a protein damage score for the protein represented by the interactive element.

21. The system of claim 20, the instructions further comprising generating a color graph mapping a range of protein damage scores to a range of colors, and wherein the indication for the interactive element is a respective color for the protein damage score for the interactive element obtained from the color graph.

22. The system of claim 16, wherein the set of anatomical organs involved in the PK or PD pathway are anatomical organs related to administration, transportation, or metabolism of the one or more drugs in the patient's body.

23. The system of claim 16, wherein the first screen further displays another set of interactive elements representing the one or more drugs or metabolites generated from the one or more drugs, wherein each interactive element is overlaid on the anatomical organ where the PK or PD pathway involving the one or more drugs of the interactive element occur.

24. The system of claim 23, wherein the first screen further comprises a directed edge from a first interactive element representing a drug overlaid on a first anatomical organ where the drug is administered to a second interactive element representing the drug overlaid on a second anatomical organ where the drug is transported or metabolized.

25. The system of claim 16, wherein responsive to receiving another request from the client device, generating a second screen of the interactive user interface, the second screen displaying:
a color graph mapping a range of drug safety scores to a range of colors, and
an indication marking the drug safety scores of the one or more drugs for the patient on the color graph.

26. The system of claim 16, wherein a protein damage score for a respective protein is computed from one or more genome sequence variation scores for one or more genome sequence variations in the patient, each genome sequence variation score indicating a degree of genome sequence variation in the patient that causes change to structure and/or function of the respective protein.

27. The system of claim 16, further comprising, responsive to receiving another request from the client device, generating a second screen of the interactive user interface, the second screen displaying:
a line graph mapping the drug safety scores of the one or more drugs for the patient, wherein the one or more drugs have a same classification under anatomical therapeutical classification (ATC) system.

28. The system of claim 16, wherein the first screen further displays a visual depiction of protein damage scores, drug safety scores or their distribution in a population obtained using gene sequence information of the population.

29. The system of claim 28, wherein the first screen further displays a visual representation of the protein damage scores or the drug safety scores of the patient, relative to the protein damage scores, drug safety scores, or their distribution in the population.

30. The system of claim 28, wherein the population represents an ethnic, racial, gender, or age group.
